(19) United States
(12) Patent Application Publication
(10) Pub. No.: US 2024/0025966 A1
(43) Pub. Date: Jan. 25, 2024

(54) CD47 BLOCKADE AND COMBINATION THERAPIES THEREOF FOR REDUCTION OF VASCULAR INFLAMMATION

(71) Applicant: The Board of Trustees of the Leland Stanford Junior University, Stanford, CA (US)

(72) Inventors: Nicholas J. Leeper, Stanford, CA (US); Irving L. Weissman, Stanford, CA (US); Kai-Uwe Jarr, Redwood City, CA (US); Yoko Kojima, Redwood City, CA (US)

(21) Appl. No.: 18/031,840

(22) PCT Filed: Oct. 21, 2021
(86) PCT No.: PCT/US2021/056090

§ 371 Date: Apr. 13, 2023

Related U.S. Application Data
(60) Provisional application No. 63/106,794, filed on Oct. 28, 2020.

(51) Int. Cl. C07K 14/705; G01N 33/68; A61P 29/00

(52) U.S. Cl. CPC C07K 14/70596; G01N 33/6893; A61P 29/00; C07K 2319/30; G01N 2800/52

(57) ABSTRACT

Methods are provided for the prevention and treatment of vascular inflammation. The methods comprise administering to a human subject an effective dose of an agent that specifically binds to CD47, and reduces one or more indicia of vascular inflammation.

### CROSS-REFERENCE TO RELATED APPLICATIONS

This application is a 371 and claims the benefit of PCT Application No. PCT/US2021/056090, filed Oct. 21, 2021, which claims the benefit of U.S. provisional Patent Application Ser. No. 63/106,794, filed on Oct. 28, 2020, the contents of which are herein incorporated by reference in their entirety.

### FEDERALLY SPONSORED RESEARCH AND DEVELOPMENT

This invention was made with government support under HL144475 awarded by the National Institutes of Health. The government has certain rights in the invention.

### INCORPORATION BY REFERENCE OF SEQUENCE LISTING

A Sequence Listing is provided herewith as a Sequence Listing text, S20-433_STAN-1801_SeqListing_ST25” created on Apr. 13, 2023, and having a size of 44,987 bytes. The contents of the Sequence Listing text are incorporated herein by reference in their entirety.

### BACKGROUND

Atherosclerosis, the leading cause of cardiovascular (CV)-related deaths worldwide, is a disease process that is initiated, maintained and destabilized by an abnormal engagement of several cellular and molecular pathways of the inflammation cascade. Exposure to elevated plasma low-density lipoprotein (LDL) cholesterol levels, either in the presence of or in the absence of additional CV risk factors, initiates and drives progressive lipid and inflammatory cell infiltration in the arterial wall, which may result in atherosclerotic plaque complications (e.g., erosion, rupture, etc.), ischemic-related organ injury and death.

In general, atherosclerosis is believed to be a complex disease involving multiple biological pathways. Variations in the natural history of the atherosclerotic disease process, as well as differential response to risk factors and variations in the individual response to therapy, reflect in part differences in genetic background and their intricate interactions with the environmental factors that are responsible for the initiation and modification of the disease. Atherosclerotic disease is also influenced by the complex nature of the cardiovascular system itself where anatomy, function and biology all play important roles in health as well as disease.

### SUMMARY

The present disclosure relates, at least in part, to methods and compositions for reducing vascular inflammation in a subject. The disclosure is based, at least in part, upon the discovery that pro-efferocytic therapies (for example, an anti-CD47 agent) can be used for the treatment of vascular inflammation in a subject.

As described in Example 1, blockade of CD47 led to a reduction in arterial FDG uptake in mouse models of atherosclerosis as well as humans. These data provide the first human evidence that pro-efferocytic therapies reduce vascular inflammation in cardiovascular disease. Without being bound by theory, it is believed that reactivating macrophage phagocytosis can clear inflamed and apoptotic tissue from the plaque and reduce lesion vulnerability.

In other aspects, the present disclosure provides, at least in part, that pro-efferocytic therapies amplify the benefits of statins (Example 3). This observed benefit occurs independent of classical risk factors like hypertension, glucose, and lipid levels. Without being bound by theory, it is believed that because pro-phagocytic therapies reduce risk irrespective of traditional risk pathways, reactivating intraplaque efferocytosis is a target for the residual inflammatory risk in atherosclerosis. In some embodiments, reactivating efferocytosis can be accomplished by targeting either CD47 or SIRPα's downstream effector molecule, SHP-1.

The data provided in Example 3 provide evidence that atorvastatin promotes efferocytosis via a reduction in CD47, leading to a lipid-independent anti-atherosclerotic effect. The combination of CD47-SIRPα blockade and HMG-CoA reductase inhibition amplifies the phagocytic capacity of macrophages and thus prevents necrotic core expansion in an additive manner.

Methods are provided for the prevention and treatment of coronary artery disease (CAD) in a subject including, without limitation, methods of reducing vascular inflammation. The methods comprise administering to a human subject an effective dose of an agent that specifically binds to CD47, and reduces one or more indicia of vascular inflammation. In some embodiments, the methods comprise administering a combination of an agent that specifically binds to CD47, e.g. an anti-CD47 antibody, with an effective dose of a statin. In some embodiments, the methods comprise administering a combination of a soluble high affinity SIRPα protein (interchangeably referred to herein as “SIRPα polypeptide” or “SIRPα reagent”) that specifically binds to CD47, with an effective dose of a statin. In some embodiments, the combination therapy provides for a synergistic effect, relative to the effect of the antibody or the statin administered as a monotherapy. In some embodiments, the combination therapy provides for an additive effect, relative to the effect of the antibody or the statin administered as a monotherapy. In some such embodiments, the methods are performed in the absence of genetic testing of the subject for the presence of a 9p21 risk allele.

An anti-CD47 agent for use in the methods of the invention interferes with binding between CD47 present on a cell and SIRPα present on a phagocytic cell. Such methods decrease vascular inflammation. In some embodiments, suitable anti-CD47 agents include without limitation soluble SIRPα polypeptides and anti-CD47 antibodies, where the term antibody encompasses antibody fragments and variants thereof, as known in the art. In some embodiments the anti-CD47 agent is an anti-CD47 antibody. In some embodiments the anti-CD47 antibody is a non-hemolytic antibody. In some embodiments the antibody comprises a human IgG4 Fc region. In some embodiments, an anti-CD47 agent is a soluble SIRPα polypeptide. In some embodiments, a soluble SIRPα polypeptide includes, without limitation, a high affinity variant SIRPα peptide fused to a human Fc region sequence. In some embodiments, the Fc region sequence is an active human Fc sequence, e.g. IgG4 Fc. In some embodiments, the high affinity SIRPα agent is CV1-hIgG4, FD6-hIgG4, etc.

In some embodiments, treatment may comprise administering a synergistic combination of an anti-CD47 agent and one or more statins. In some embodiments, treatment comprises administering an additive combination of an anti-CD47 agent and one or more statins. It is shown herein that statins, e.g. atorvastatin, augment efferocytosis by inhibiting the nuclear translocation of NFκB1 p50 and suppressing expression of CD47. In some embodiments, statins of interest for the methods disclosed herein include, without limitation, atorvastatin, fluvastatin, lovastatin, mevastatin, pitavastatin, pravastatin, rosuvastatin, simvastatin, etc. In some embodiments, a high affinity SIRPα agent is administered in combination with atorvastatin. The active agents of the combination may be administered separately.

The agents in a combination are administered concomitantly, i.e. where a statin is administered as generally prescribed, e.g. daily, and the anti-CD47 agent is administered at suitable intervals, e.g. every 2 weeks, weekly, semi-weekly, etc. The agents can be considered to be combined if administration scheduling is such that the serum levels of both agents are concomitantly at a therapeutic level. A benefit of the present invention can be the use of lowered doses of one or both of the agents relative to the dose required as a monotherapy, providing a reduction in undesirable side effects, e.g. anemia associated with anti-CD47 agents; muscle pain, liver damage and hyperglycemia associated with statin use, etc.

In some embodiments the methods of treatment further comprise monitoring the level of vascular inflammation in a subject. In some embodiments an individual is treated according to the results of such an assessment, e.g. therapy is continued or discontinued based on the results, or where dosages are altered according to the results. In some embodiments the indicia of vascular inflammation a change in vascular ¹⁸F-FDG uptake. Such changes may be monitored as known in the art, e.g. by PET/CT scanning.

In some embodiments a primer agent is administered prior to administering a therapeutically effective dose of an anti-CD47 agent to the individual. Suitable primer agents include an erythropoiesis-stimulating agent (ESA), and/or a priming (sub-therapeutic) dose of an anti-CD47 agent. Following administration of the priming agent, and allowing a period of time effective for an increase in reticulocyte production, a therapeutic dose of an anti-CD47 agent is administered. The therapeutic dose can be administered in number of different ways. In some embodiments, two or more therapeutically effective doses are administered after a primer agent is administered. In some embodiments a therapeutically effective dose of an anti-CD47 agent is administered as two or more doses of escalating concentration, in others the doses are equivalent.

In some aspects, the disclosure provides a method of reducing vascular inflammation in a human subject, the method comprising administering to the subject an effective dose of an anti-CD47 agent; and monitoring the subject for indicia of vascular inflammation.

In some embodiments, method is performed in the absence of genotyping the subject for the presence of at least one 9p21 risk allele.

In some embodiments, the anti-CD47 agent specifically binds to CD47. In some embodiments, the anti-CD47 agent is an antibody. In some embodiments, antibody comprises an IgG4 constant region. In some embodiments, the antibody does not activate CD47 upon binding.

In some embodiments, the anti-CD47 agent is a soluble SIRPα polypeptide. In some embodiments, the soluble SIRPα polypeptide comprises an immunoglobulin constant region. In some embodiments, the soluble SIRPα polypeptide is multimerized through the immunoglobulin constant region. In some embodiments, the SIRPα polypeptide is selected from a CV1-hIgG4, CV1 monomer, FD6-hIgG4 or a FD6 monomer. In some embodiments, the SIRPα polypeptide is selected from the polypeptides in Table 1. In some embodiments, the SIRPα polypeptide is selected from SEQ ID NOs: 1-17.

In some embodiments, the anti-CD47 agent is administered to the subject at a dose of 20-45 mg/kg weekly. In some embodiments, the anti-CD47 agent is administered to the subject weekly for at least nine weeks.

In some embodiments, the method further comprises administering a priming dose of the anti-CD47 agent to the subject prior to administering the therapeutically effective dose of the anti-CD47 agent to the subject. In some embodiments, the priming dose is administered to the subject at a dose of 1 mg/kg.

In some embodiments, vascular inflammation is reduced by at least 10%. In some embodiments, vascular inflammation is reduced by at least 20%.

In some embodiments, the indicia of vascular inflammation is a change in vascular ¹⁸F-FDG uptake, high sensitivity C-reactive protein (hsCRP), C-reactive protein (CRP), IL-6, IL-8, fibrinogen, Human serum amyloid A (SAA), Haptoglobin (Hp), secretory phospholipase A2 (sPLA2), Lipoprotein(a), apolipoprotein B (APOB) to apolipoprotein A1 (APOA1) ratio, and/or white blood cell count (WBC).

In some embodiments, the indicia of vascular inflammation is a change in vascular ¹⁸F-FDG uptake. In some embodiments, ¹⁸F-FDG uptake is reduced by at least 10%. In some embodiments, ¹⁸F-FDG uptake is reduced by at least 20%. In some embodiments, ¹⁸F-FDG uptake is reduced as measured by maximum standardized update values (SUV) and/or maximum target-to-background ratio (TBR). In some embodiments, the change in vascular ¹⁸F-FDG uptake is monitored by combined Positron Emission Tomography (PET) and computed tomography (CT).

In some aspects, the disclosure provides a method of reducing vascular inflammation in a human subject, the method comprising administering to the subject an effective dose of an anti-CD47 agent in combination with an effective dose of a statin, wherein the combination provides fo a reduction in vascular inflammation relative to the effect of either agent as a monotherapy.

In some embodiments, the reduction in vascular inflammation is additive relative to the effect of either agent as a monotherapy. In some embodiments the reduction in vascular inflammation is synergistic relative to the effect of either agent as a monotherapy.

In some embodiments, the statin is selected from atorvastatin, fluvastatin, lovastatin, mevastatin, pitavastatin, pravastatin, rosuvastatin, and simvastatin.

In some embodiments, the method is performed in the absence of genotyping the subject for the presence of at least one 9p21 risk allele.

In some embodiments, the anti-CD47 agent specifically binds to CD47. In some embodiments the anti-CD47 agent is an antibody. In some embodiments the antibody comprises an IgG4 constant region. In some embodiments the antibody does not activate CD47 upon binding.

In some embodiments, the anti-CD47 agent is a soluble SIRPα polypeptide. In some embodiments the soluble SIRPα polypeptide comprises an immunoglobulin constant region. In some embodiments the soluble SIRPα polypeptide is multimerized through the immunoglobulin constant region. In some embodiments, the SIRPα polypeptide is selected from a CV1-hIgG4, CV1 monomer, FD6-hIgG4 or a FD6 monomer. In some embodiments, the SIRPα polypeptide is selected from the polypeptides in Table 3. In some embodiments, the SIRPα polypeptide is selected from SEQ ID NOs: 1-17.

In some embodiments, the reduction in vascular inflammation results in a plaque area as a measure of total vessel area is reduced by at least 5% compared to the absence of intervention.

In some embodiments, the reduction in vascular inflammation results in a necrotic core as a measure of the percentage of intima area is reduced by at least 5% compared to the absence of intervention.

In some embodiments, the reduction in vascular inflammation results in an increased rate of efferocytosis. In some embodiments, the rate of efferocytosis is increased by at least 10% compared to the absence of intervention.

### BRIEF DESCRIPTION OF THE DRAWINGS

FIGS. 1A-FIG. 1D. Reduction of vascular 18F-FDG uptake in mice after anti-CD47 therapy. Panel A shows representative PET/CT images of carotid 18F-FDG uptake (sagittal view). Voxels of interest are drawn upstream (caudally) from the cast. Arrows indicate the carotid artery. Panel B shows the quantification of the carotid 18F-FDG uptake. Reduced vascular signal measured by mean SUV was observed in anti-CD47 treated animals compared to controls. Panel C shows representative hematoxylin and eosin images demonstrating carotid plaque size according to the treatment received. Panel D shows the quantification of carotid plaque size for validation of PET/CT results. Panel E shows representative PET/CT images of aortic 18F-FDG uptake (coronal and axial views). Arrows indicate the aorta. Panel F shows the quantification of the aortic 18F-FDG uptake measured by mean SUV. Reduced vascular signal inflammation was noted as early as six weeks after treatment initiation. Data are presented as mean and standard deviation and were analyzed using an unpaired t-test (two-tailed) or a Mann-Whitney test (two-tailed).

FIGS. 2A-FIG. 2E Reduction of vascular 18F-FDG uptake in patients after magrolimab therapy. Panel A demonstrates a reduction in vascular 18F-FDG uptake measured by maximum SUV and TBR in the most diseased segment of the index vessel after magrolimab therapy. Panel B shows a waterfall plot of maximum TBR (in percent change from baseline) in all nine patients, according to the maintenance dose received. Of note, patient number 7 (black asterisk) was initiated at a maintenance dose of 45 mg per kilogram body weight but was later changed to 30 mg per kilogram. Panel C, D, and E show representative 18F-FDG-PET/CT scans (axial view) of the middle tertile of patients (patient number 4, number 5, and number 6). Arrows indicate the index vessel (carotid artery) at baseline and post magrolimab. Data are presented as mean and standard deviation and were analyzed using a paired t-test (two-tailed).

FIG. 3. RNA sequencing analysis revealed lovastatin as one of the top upstream regulators of the CD47/SIRP-alpha axis in macrophages in vitro. RNA sequencing was performed on bone marrow-derived mouse macrophages treated with a nanoparticle loaded with a chemical inhibitor of the Src homology 2 domain-containing phosphatase-1 (SHP-1) and thus interrupting the CD47/SIRP-alpha signaling axis. A, The Volcano-Plot shows genes which were differentially expressed by the SHP-1 inhibition. B, Using Ingenuity Pathway Analysis (Qiagen), lovastatin was one of the top upstream regulators, suggesting overlapping mechanism of action between the interruption of the CD47/SIRPalpha axis and statin signaling and thus additive effects on macrophages in preventing atherosclerosis.

FIGS. 4A-FIG. 4B. The combination of pro-efferocytic therapies (anti-CD47 antibodies or nanoparticles loaded with a SHP1-inibitor) and atorvastatin treatment showed additive effects on atherosclerotic plaque burden in vivo. Atheroprone apolipoprotein-E-deficient mice were treated with (1) IgG isotype control antibodies, (2) anti-CD47 antibodies, (3) atorvastatin, (4) the combination of anti-CD47 antibodies and atorvastatin, and (5) the combination of the nanoparticle loaded with a SHP1-inhibitor and atorvastatin. FIG. 4A, Additive effects on plaque burden (measured as plaque area in % of total vessel area) in mice treated with the combination of pro-efferocytic therapies and atorvastatin were observed. FIG. 4B, Additionally, the necrotic core size (measured as necrotic core in % of intima area) was significantly reduced in the cohorts treated with a combined regimen.

FIGS. 5A-FIG. 5C. RNA sequencing revealed HMG-CoA reductase inhibitor as one of the top upstream regulators of SHP-1 inhibition in macrophages. FIG. 5A [[a]], Volcano plot of genes that regulate the response to SHP1i in bone marrow-derived macrophages (n=3 biological replicates per group). Significant hits were defined by a false-discovery rate <0.10 and marked as (downregulated) or (upregulated). FC, fold change; Rbl1, RB transcriptional corepressor like 1; Xiap, X-linked inhibitor of apoptosis; Apoe, apolipoprotein E; Rhob, ras homolog family member B; Gpx3, glutathione peroxidase 3. FIGS. 5B-C, Lovastatin, a first generation HMG-CoA reductase inhibitor, was one of the top activated upstream regulators and the only drug in the database, based on the relevant regulation of Apoe, Rhob, Rbl1, Gpx3, and Xiap. Filter criteria: top four upstream regulators with significant Z-score (³ 2 for predicted activation and £−2 for predicted inhibition). Sorting criteria: P value of overlap. All false-discovery rate values are provided. All significant upstream regulators are provided in Table 2.

FIGS. 6A-FIG. 6E. Combined treatment of CD47-SIRPα blockade and atorvastatin showed additive effects on atherosclerotic plaque activity in vivo. FIG. 6a, Quantification of atherosclerotic lesion area and cross-sections of aortic roots stained with Oil-red O (n=10 for Statin; n=13 for anti-CD47+Statin; n=15 for SHP1i+Statin). TVA, total vessel area; Scale bar, 100 μm. FIG. 6b, Quantification of necrotic core size and cross-sections of aortic roots stained with Masson's trichrome (n=10 for Statin; n=13 for anti-CD47+Statin; n=15 for SHP1i+Statin). Scale bar and scale bar inset, 100 μm. c, Quantification of total cholesterol, high-density lipoprotein (HDL), low-density lipoprotein (LDL), and glucose in the blood (n=7 for Statin; n=10 for anti-CD47+Statin; 10 n=12 for SHP1i+Statin). FIG. 6d-e, Applying the Bliss independence model on the analyses of lesion area and necrotic core size to determine additivity/synergy of compounds (n=10 for Ecalculated; n=13 for anti-CD47+Statin Eobserved; n=15 for SHP1i+Statin Eobserved). Δ, change in. Each data point represents a biological replicate. Data and error bars present the mean+/−standard error of the mean. Data of (a) were analysed by one-way analysis of variance with Tukey's multiple comparisons test. Data of (b-c) were analysed by Kruskal-Wallis with Dunn's multiple comparisons test. Data of (d) were analysed by unpaired Student's t-test and unpaired Welch's t-test (two-tailed). Data of (e) were analyzed by unpaired Student's t-test (two-tailed) and Mann-Whitney U test (two-tailed). All p values are provided in Source Data.

FIGS. 7A-FIG. 7F. Combined treatment of CD47-SIRPα blockade and atorvastatin showed additive effects on efferocytosis rate in vitro and in vivo. FIG. 7a, Quantification of efferocytosis rate and flow cytometry plots depicting the efferocytosis rate in vitro in the presence or absence of atorvastatin, SHP1i, and dual treatment (n=3 biological replicates per group). The right upper quadrant (highlighted in red) includes double-positive cells that are taken to represent a macrophage that has ingested an apoptotic target cell. FIG. 7b, Applying the Bliss independence model on the analyses of efferocytosis rate in vitro to determine additivity/synergy of compounds (n=3 biological replicates per group). FIG. 7c, Apoptosis assay to quantify the rate of programmed cell death in vitro in the presence or absence of atorvastatin, SHP1i, and dual treatment (n=5 biological replicates per group). Rel., relative. W/o, without. STS, staurosporine. FIG. 7d, Quantification of cleaved caspase-3 activity and immunofluorescence images (n=9 for PBS; n=10 for Statin; n=11 for SHP1i; n=15 for SHP1i+Statin). White line depicts intima. Scale bar, 10 μm. FIG. 7e, Quantification of efferocytosis rate in vivo and immunofluorescence images depicting the ratio of free to macrophage associated cleaved caspase-3 activity (n=9 for PBS; n=10 for Statin; n=11 for SHP1i; n=15 for SHP1i+Statin). White line depicts intima. *, free cleaved caspase-3. #, macrophage18 associated cleaved caspase-3. Scale bar, 10 μm. FIG. 7f, Applying the Bliss independence model on the analyses of efferocytosis rate in vivo to determine additivity/synergy of compounds (n=10 Ecalculated; n=15 Eobserved). Δ, change in. Each data point represents a biological replicate. Data and error bars present the mean+/−standard error of the mean. Data of (a) and (c-e) were analysed by Kruskal-Wallis with Dunn's multiple comparisons test. Data of (b) and (f) 1 were analysed by Mann-Whitney U test (two-tailed). All p values are provided in Source Data.

FIGS. 8A-FIG. 8G. Atorvastatin inhibited NFκB1 p50 nuclear translocation under atherogenic conditions and thus directly regulated gene expression of Cd47. FIG. 8a, Cd47 expression by quantitative polymerase chain reaction in smooth muscle cells (n=12 biological replicates per group). TNF-α, tumor necrosis factor-α. FIG. 8b, Cd47 expression by flow cytometry in smooth muscle cells (n=6 biological replicates per group). RFI, ratio of median fluorescence intensity. Rel., relative. Resp., respective. FIG. 8c, Cd47 expression by immunofluorescence in smooth muscle cells (n=3 biological replicates per group). AU, arbitrary unit. SMC, smooth muscle cells. Scale bar, 10 μm. FIG. 8d, Cd47 promoter activity by luciferase assay in smooth muscle cells (n=18 biological replicates per group). Rel., relative. FIG. 8e, NFκB1 p50 nuclear translocation by immunofluorescence in smooth muscle cells. NFκB1, nuclear factor of kappa light polypeptide gene enhancer in B cells 1. M, mevalonate. Scale bar, 10 μm. FIG. 8f, NFκB1 p50 nuclear translocation by Western blot in smooth muscle cells (n=11 biological replicates per group). HDAC1, histone deacetylase 1. Lane 1, Vehicle. Lane 2, TNF-α. Lane 3, TNF-α+Statin. Lane 4, TNF-α+Statin+M. FIG. 8g, CD47 expression by quantitative polymerase chain reaction in carotid endarterectomy samples (n=7 biological replicates per group). Each data point represents a biological replicate, except for (c), which shows technical replicates (mean value per high power field) of three biological replicates. Data and error bars present the mean+/−standard error of the mean. Data of (a-f) were analyzed by Kruskal-Wallis with Dunn's multiple comparisons test. Data of (g) were analyzed by Mann-Whitney U test (two-tailed). All p values are provided in Source Data.

FIG. 9. The pleiotropic benefits of statins include the activation of efferocytosis in atherosclerosis. Atorvastatin augments efferocytosis by inhibiting the nuclear translocation of NFκB1 p50 and suppressing expression of the key “don't eat me” molecule CD47. Combination of HMG-CoA reductase inhibition and CD47-SIRPα blockade amplifies the phagocytic capacity of macrophages and thus prevents atherosclerosis in an additive manner.

FIGS. 10A-FIG. 10C: RNA sequencing revealed HMG-CoA reductase inhibitor as one of the top upstream regulators of SHP-1 inhibition in macrophages. FIG. 10a, Flow cytometry gating strategy for cell sorting to isolate Cy5.5-positive bone marrow-derived macrophages in each group (SHP1i versus SWNT), which were then subjected to RNA sequencing. FIG. 10b, Rbl1, Xiap, Apoe, Rhob, and Gpx3 expression by quantitative polymerase chain reaction in bone marrow-derived macrophages upon atorvastatin treatment (n=6 biological replicates). FIG. 10c, Functional pathways enriched among all differential expressed genes (false-discovery rate <0.10) as determined by pathway analysis. Each data point represents a biological replicate. Data and error bars present the mean+/−standard error of the mean. Data of (C) were analyzed by Mann-Whitney U test (two-tailed).

FIGS. 11A-FIG. 11K: Combined treatment of CD47-SIRPα blockade and atorvastatin showed additive effects on atherosclerotic plaque activity in vivo. FIG. 11a, Quantification of atherosclerotic lesion area (n=9 for PBS; n=10 for Statin). TVA, total vessel area. FIG. 11b, Quantification of necrotic core size (n=9 for PBS; n=10 for Statin). FIG. 11c, Quantification of total cholesterol, high-density lipoprotein (HDL), low-density lipoprotein (LDL), and glucose in the blood (n=9 for PBS; n=7 for Statin). FIG. 11d, Quantification of atherosclerotic lesion area (n=13 for IgG; n=13 for anti-CD47; n=13 for anti-CD47+Statin). FIG. 11e, Quantification of necrotic core size (n=13 for IgG; n=13 for anti-CD47; n=13 for anti-CD47+Statin). FIG. 11f, Quantification of total cholesterol, high-density lipoprotein, low-density lipoprotein, and glucose in the blood (n=10 for IgG; n=11 for anti-CD47; n=10 for anti-CD47+Statin). FIG. 11g, Quantification of atherosclerotic lesion area (n=12 for SWNT; n=11 for SHP1i; n=15 for SHP1i+Statin). FIG. 11h, Quantification of necrotic core size (n=12 for SWNT; n=11 for SHP1i; n=15 for SHP1i+Statin). FIG. 11i, Quantification of total cholesterol, high-density lipoprotein, low-density lipoprotein, and glucose in the blood (n=11 for SWNT; n=10 for SHP1i; n=12 for SHP1i+Statin). FIG. 11j-k, Quantification of atherosclerotic lesion area and necrotic core size (n=10 for Statin; n=13 for anti-CD47; n=13 for anti-CD47+Statin; n=11 for SHP1i; n=15 for SHP1i+Statin). Each data point represents a biological replicate. Data and error bars present the mean+/−standard error of the mean. Data of (A) were analyzed by unpaired Welch's t-test (two-tailed). Data of (B-C) were analyzed by Mann-Whitney U test (two-tailed). Data of (D) and (G) were analyzed by one-way analysis of variance with Tukey's multiple comparisons test. Data of (E-F) and (H-I) were analyzed by Kruskal-Wallis with Dunn's multiple comparisons test. Data of (J-K) were analyzed by one-way analysis of variance with Tukey's multiple comparisons test and Kruskal-Wallis with Dunn's multiple comparisons test. All p values are provided in the Source data.

FIGS. 12A-FIG. 12D. Combined treatment of CD47-SIRPα blockade and atorvastatin showed additive effects on efferocytosis rate in vitro and in vivo. FIG. 12a, Flow cytometry plots depicting the staining controls for the conditions. FIG. 12b, Apoptosis assay to quantify the rate of programmed cell death in vitro in the presence or absence of atorvastatin, SHP1i, and dual treatment after staurosporine (STS) stimulation. W/o, without. FIG. 12c, Immunofluorescence images depicting cleaved caspase-3 activity. White line depicts intima. Scale bar, 50 μm; scale bar inset, 10 μm. FIG. 12d, Immunofluorescence images depicting the ratio of free to macrophage associated cleaved caspase-3 activity. White line depicts intima. Scale bar, 10 μm. Each data point represents a biological replicate. Data and error bars present the mean+/−standard error of the mean. Data of (B) were analyzed by Kruskal-Wallis with Dunn's multiple comparisons test. All p values are provided in the Source data.

FIGS. 13A-FIG. 13D. Atorvastatin inhibited NFκB1 p50 nuclear translocation under atherogenic conditions and thus directly regulated gene expression of CD47. FIG. 13a, Cd47 expression by quantitative polymerase chain reaction in bone marrow-derived macrophages (n=6 biological replicates). TNF-α, tumor necrosis factor-α. FIG. 13b, Cd47 expression by flow cytometry in bone marrow-derived macrophages (n=4 biological replicates). RFI, ratio of median fluorescence intensity. Rel., relative. Resp., respective. FIG. 13c, Cd47 expression by immunofluorescence in smooth muscle cells. SMC, smooth muscle cells. Scale bar, 10 μm. FIG. 13d, NFκB1 p50 nuclear translocation by immunofluorescence in smooth muscle cells. NFκB1, nuclear factor of kappa light polypeptide gene enhancer in B cells 1. M, mevalonate. Scale bar and scale bar inset, 10 μm. Each data point of represents a biological replicate. Data and error bars present the mean+/−standard error of the mean. Data of (A-B) were analyzed by Kruskal-Wallis with Dunn's multiple comparisons test. All p values are provided in the Source data.

### DETAILED DESCRIPTION OF THE INVENTION

Coronary artery disease (CAD) is a narrowing or blockage of the arteries and vessels that provide oxygen and nutrients to the heart. It is associated with vascular inflammation, which causes atherosclerosis, an accumulation of fatty materials on the inner linings of arteries. The resulting blockage restricts blood flow to the heart. When the blood flow is completely cut off, the result is a heart attack. CAD is the leading cause of death for both men and women in the United States.

Atherosclerosis (also referred to as arteriosclerosis, atheromatous vascular disease, arterial occlusive disease) as used herein, refers to a cardiovascular disease characterized by plaque accumulation on vessel walls and vascular inflammation. The plaque consists of accumulated intracellular and extracellular lipids, smooth muscle cells, connective tissue, inflammatory cells, and glycosaminoglycans. Vascular inflammation occurs in combination with lipid accumulation in the vessel wall, and vascular inflammation is a hallmark of the atherosclerosis disease process.

Myocardial infarction is an ischemic myocardial necrosis usually resulting from abrupt reduction in coronary blood flow to a segment of myocardium. In the great majority of patients with acute MI, an acute thrombus, often associated with plaque rupture, occludes the artery that supplies the damaged area. Plaque rupture occurs generally in vessels previously partially obstructed by an atherosclerotic plaque enriched in inflammatory cells. Altered platelet function induced by endothelial dysfunction and vascular inflammation in the atherosclerotic plaque presumably contributes to thrombogenesis. Myocardial infarction can be classified into ST-elevation and non-ST elevation MI (also referred to as unstable angina). In both forms of myocardial infarction, there is myocardial necrosis. In ST-elevation myocardial infraction there is transmural myocardial injury which leads to ST-elevations on electrocardiogram. In non-ST elevation myocardial infarction, the injury is sub-endocardial and is not associated with ST segment elevation on electrocardiogram. Myocardial infarction (both ST and non-ST elevation) represents an unstable form of atherosclerotic cardiovascular disease. Acute coronary syndrome encompasses all forms of unstable coronary artery disease. Heart failure can occur as a result of myocardial dysfunction caused by myocardial infraction.

Angina refers to chest pain or discomfort resulting from inadequate blood flow to the heart. Angina can be a symptom of atherosclerotic cardiovascular disease. Angina may be classified as stable, which follows a regular chronic pattern of symptoms, unlike the unstable forms of atherosclerotic vascular disease. The pathophysiological basis of stable atherosclerotic cardiovascular disease is also complicated but is biologically distinct from the unstable form. Generally stable angina is not myocardial necrosis.

Measurements of vascular disease include, for example, echocardiography and angiography, which have traditionally been the primary imaging modalities for diagnosing cardiac disease. Computed tomography (CT) and magnetic resonance (MR) imaging are used with increasing frequency because they improve tissue characterization. Intravascular ultrasonography, CT, and MR imaging are frequently used to detect atherosclerotic plaque, wall thickening, and luminal stenosis or enlargement; quantify the extent of the disease; and identify complications such as aneurysm, dissection, and thrombus.

Diagnostic tools that are more directly reflective of vascular inflammation have been sought. Positron emission tomography (PET) performed with fluorine 18 fluorodeoxyglucose (FDG) has the unique ability to depict metabolically active disease, and in this respect, it complements other cross-sectional imaging modalities, which provide predominantly anatomic information. Because whole-body imaging with combined Positron Emission Tomography (18F-FDG PET) combined with computed tomography (CT) (hereafter, PET/CT) is used with increasing frequency to evaluate noncardiovascular disease processes, it may be the first imaging study in which cardiovascular disease is identified. FDG PET/CT has become a valuable imaging modality for diagnosing various conditions in patients who present with systemic symptoms that are difficult to localize and diagnose with a clinical examination and routine imaging procedures. These methods can be used in both preclinical and clinical studies for the evaluation of inflammation in the arterial wall. Technical progress to extend the CV applications of ¹⁸F-FDG PET/CT include improved image acquisition, measurements, and reconstruction protocols. This has allowed a number of clinical trials to provide results of ¹⁸F-FDG PET/CT in detecting atherosclerotic plaque inflammation, discriminating stable from unstable plaques, predicting CV prognosis, and monitoring response to CV-related therapies.

¹⁸F-FDG PET has been used to assess the impact of statin treatment on arterial wall inflammation in interventional studies. For this purpose, arterial ¹⁸F-FDG uptake is expressed as the Target-to-Background Ratio (TBR), that is a measure of the blood-normalized standardized uptake value (SUV). ¹⁸F-FDG is taken up mostly by macrophages within the atherosclerotic plaques, although other cells (i.e., endothelial cells, vascular smooth muscle cells, neutrophils, lymphocytes) may participate in tracer uptake. TBR, as a measure of SUV, has been demonstrated to be a reproducible index for quantification of ¹⁸F-FDG uptake in the inflamed arterial wall. While many atherosclerotic plaques are not metabolically active at FDG PET, focal intense activity within atherosclerotic plaques may be a marker of lesions that are vulnerable to disruption and have more inflammatory cellular components.

In some embodiments, efficacy of a combination therapy disclosed herein is monitored by ¹⁸F-FDG PET/CT, including specifically the determination of TBR as a function of SUV, where decreased uptake, e.g. up to about 5% decrease, up to about 10% decrease, up to about 25% decrease, up to about 50% decrease, or more, is indicative of therapeutic efficacy.

In some embodiments, efficacy of an anti-CD47 agent disclosed herein is monitored by ¹⁸F-FDG PET/CT, including specifically the determination of TBR as a function of SUV, where decreased uptake, e.g. up to about 5% decrease, up to about 10% decrease, up to about 25% decrease, up to about 50% decrease, or more, is indicative of therapeutic efficacy.

Anti-CD47 agent. CD47 is a broadly expressed transmembrane glycoprotein with a single Ig-like domain and five membrane spanning regions, which functions as a cellular ligand for SIRPα with binding mediated through the NH2-terminal V-like domain of SIRPα. SIRPα is expressed primarily on myeloid cells, including macrophages, granulocytes, myeloid dendritic cells (DCs), mast cells, and their precursors, including hematopoietic stem cells. Structural determinants on SIRPα that mediate CD47 binding are discussed by Lee et al. (2007) J. Immunol. 179:7741-7750; Hatherley et al. (2008) Mol Cell. 31(2):266-77; Hatherley et al. (2007) J. B. C. 282:14567-75; and the role of SIRPα cis dimerization in CD47 binding is discussed by Lee et al. (2010) J. B. C. 285:37953-63.

As used herein, the term “anti-CD47 agent” or “agent that provides for CD47 blockade” refers to an agent that reduces the binding of CD47 (e.g., on a target cell) to SIRPα (e.g., on a phagocytic cell). Non-limiting examples of suitable anti-CD47 agents include SIRPα reagents, including without limitation high affinity SIRPα polypeptides, and anti-CD47 antibodies or antibody fragments. In some embodiments, an anti-CD47 agent of the disclosure is an anti-CD47 antibody, or an antigen binding fragment thereof. In some embodiments, an anti-CD47 agent of the disclosure is a SIRPα polypeptide. In some embodiments, an anti-CD47 agent of the disclosure is a high affinity SIRPα polypeptide. In some embodiments, a suitable anti-CD47 agent e.g. an anti-CD47 antibody, a SIRPα reagent, etc., specifically binds CD47 to reduce the binding of CD47 to SIRPα. A suitable anti-CD47 agent that binds SIRPα does not activate SIRPα (e.g., in the SIRPα-expressing phagocytic cell). The efficacy of a suitable anti-CD47 agent can be assessed by assaying the agent. In an exemplary assay, target cells are incubated in the presence or absence of the candidate agent and in the presence of an effector cell, e.g. a macrophage or other phagocytic cell. An agent for use in the methods of the invention will up-regulate phagocytosis by at least 5% (e.g., at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 500%, at least 1000%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-CD47 agent does not activate CD47 upon binding. When CD47 is activated, a process akin to apoptosis (i.e., programmed cell death) may occur (Manna and Frazier, Cancer Research, 64, 1026-1036, Feb. 1, 2004). Thus, in some embodiments, the anti-CD47 agent does not directly induce cell death of a CD47-expressing cell.

In some embodiments a primer agent is administered prior to administering a therapeutically effective dose of an anti-CD47 agent to the individual. In some embodiments, a primer agent is administered at a sub-therapeutic dose of an anti-CD47 agent of the disclosure. In some embodiments, a sub-therapeutic dose may be, for example, less than about 10 mg/kg, less than about 7.5 mg/kg, less than about 5 mg/kg, less than about 2.5 mg/kg, and may be less than or about 1 mg/kg. Suitable primer agents include an erythropoiesis-stimulating agent (ESA), and/or a priming dose of an anti-CD47 agent. Following administration of the priming agent, and allowing a period of time effective for an increase in reticulocyte production, a therapeutic dose of an anti-CD47 agent is administered. Administration may be made in accordance with the methods described in U.S. Pat. No. 9,623,079, herein specifically incorporated by reference.

SIRPα reagent. A SIRPα reagent comprises the portion of SIRPα that is sufficient to bind CD47 at a recognizable affinity, which normally lies between the signal sequence and the transmembrane domain, or a fragment thereof that retains the binding activity. A suitable SIRPα reagent reduces (e.g., blocks, prevents, etc.) the interaction between the native proteins SIRPα and CD47. The SIRPα reagent will usually comprise at least the dl domain of SIRPα.

In some embodiments, an anti-CD47 agent of the disclosure is a SIRPα reagent. In some embodiments, a subject anti-CD47 agent is a “high affinity SIRPα reagent”, which includes SIRPα-derived polypeptides and analogs thereof. Specific variants of interest include, without limitation, those disclosed in U.S. Pat. No. 9,944,911, herein specifically incorporated by reference. SIRPα peptides of interest include, for example, monomers and fusions to Fc region sequences, e.g. CV1-hIgG4, CV1 monomer, FD6-hIgG4, and FD6 monomer, etc. High affinity SIRPα reagents are variants of the native SIRPα protein. The amino acid changes that provide for increased affinity are localized in the dl domain, and thus high affinity SIRPα reagents comprise a dl domain of human SIRPα, with at least one amino acid change relative to the wild-type sequence within the dl domain. Such a high affinity SIRPα reagent optionally comprises additional amino acid sequences, for example antibody Fc sequences; portions of the wild-type human SIRPα protein other than the dl domain, including without limitation residues 150 to 374 of the native protein or fragments thereof, usually fragments contiguous with the dl domain; and the like. In some embodiments, a SIRPα reagent is a SIRPα polypeptide. In some embodiments, a SIRPα reagent is a high affinity SIRPα polypeptide. In some embodiments, a SIRPα reagent is a fusion protein.

High affinity SIRPα reagents may be monomeric or multimeric, i.e. dimer, trimer, tetramer, etc., for example multimerized through an immunoglobulin Fc sequence. For instance, the variant dl domain of may be fused to an IgG, IgA or an IgD Fc domain. When the dl domain of CV1 is fused to an IgG Fc domain, the IgG subclass may be an IgG1, IgG2a, IgG2b, IgG3 or an IgG4 subclass. The Fc sequence may be an active Fc, that binds to, and activates, its cognate Fc receptor. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

In some embodiments, an anti-CD47 agent of the disclosure is a full-length signal-regulatory protein alpha (SIRP-α) protein, or portion thereof. In some embodiments, an anti-CD47 agent of the disclosure is a SIRP-α peptide sequence. In some embodiments, the anti-CD47 agent comprises or consists of the d1 domain of SIRP-α. The SIRP-α protein is also known as tyrosine-protein phosphatase non-receptor type substrate 1, CD172 antigen-like family member A, brain-immunoglobulin-like molecule with tyrosine-based activation motifs, inhibitory receptor Src-homology 2-domain bearing protein tyrosine phosphatase 1, macrophage fusion receptor, and tyrosine phosphatase Src-homology protein substrate 1.

In some embodiments, the SIRP-α peptide sequence comprises or consists of the human wild type SIRP-α sequence variant 1; NP_001035111; SEQ ID NO: 1. D1 domain in bold.

(SEQ ID NO: 1)
MEPAGPAPGRLGPLLCLLLAASCAWSGVAGEEELQVIQPDKSVLVAAGE
TATLRCTATSLIPVGPIQWFRGAGPGRELIYNQKEGHFPRVTTVSDLTK
RNNMDFSIRIGNITPADAGTYYCVKFRKGSPDDVEFKSGAGTELSVRAK
PSAPVVSGPAARATPQHTVSFTCESHGFSPRDITLKWFKNGNELSDFQT
NVDPVGESVSYSIHSTAKVVLTREDVHSQVICEVAHVTLQGDPLRGTAN
LSETIRVPPTLEVTQQPVRAENQVNVTCQVRKFYPQRLQLTWLENGNVS
RTETASTVTENKDGTYNWMSWLLVNVSAHRDDVKLTCQVEHDGQPAVSK
SHDLKVSAHPKEQGSNTAAENTGSNERNIYIVVGVVCTLLVALLMAALY
LVRIRQKKAQGSTSSTRLHEPEKNAREITQDTNDITYADLNLPKGKKPA
PQAAEPNNHTEYASIQTSPQPASEDTLTYADLDMVHLNRTPKQPAPKPE
PSFSEYASVQVPRK.

In some embodiments, the SIRP-α peptide sequence comprises or consists of the human wild type SIRP-α sequence variant 2; NP_001035112; SEQ ID NO: 2. D1 domain in bold.

(SEQ ID NO: 2)
MEPAGPAPGRLGPLLCLLLAASCAWSGVAGEEELQVIQPDKSVLVAAGE
TATLRCTATSLIPVGPIQWFRGAGPGRELIYNQKEGHFPRVTTVSDLTK
RNNMDFSIRIGNITPADAGTYYCVKFRKGSPDDVEFKSGAGTELSVRAK
PSAPVVSGPAARATPQHTVSFTCESHGFSPRDITLKWFKNGNELSDFQT
NVDPVGESVSYSIHSTAKVVLTREDVHSQVICEVAHVTLQGDPLRGTAN
LSETIRVPPTLEVTQQPVRAENQVNVTCQVRKFYPQRLQLTWLENGNVS
RTETASTVTENKDGTYNWMSWLLVNVSAHRDDVKLTCQVEHDGQPAVSK
SHDLKVSAHPKEQGSNTAAENTGSNERNIYIVVGVVCTLLVALLMAALY
LVRIRQKKAQGSTSSTRLHEPEKNAREITQDTNDITYADLNLPKGKKPA
PQAAEPNNHTEYASIQTSPQPASEDTLTYADLDMVHLNRTPKQPAPKPE
PSFSEYASVQVPRK.

In some embodiments, the SIRP-α peptide sequence comprises or consists of the human wild type SIRP-α sequence variant 3; NP_542970; SEQ ID NO: 3:

(SEQ ID NO: 3)
MEPAGPAPGRLGPLLCLLLAASCAWSGVAGEEELQVIQPDKSVLVAAGE
TATLRCTATSLIPVGPIQWFRGAGPGRELIYNQKEGHFPRVTTVSDLTK
RNNMDFSIRIGNITPADAGTYYCVKFRKGSPDDVEFKSGAGTELSVRAK
PSAPVVSGPAARATPQHTVSFTCESHGFSPRDITLKWFKNGNELSDFQT
NVDPVGESVSYSIHSTAKVVLTREDVHSQVICEVAHVTLQGDPLRGTAN
LSETIRVPPTLEVTQQPVRAENQVNVTCQVRKFYPQRLQLTWLENGNVS
RTETASTVTENKDGTYNWMSWLLVNVSAHRDDVKLTCQVEHDGQPAVSK
SHDLKVSAHPKEQGSNTAAENTGSNERNIYIVVGVVCTLLVALLMAALY
LVRIRQKKAQGSTSSTRLHEPEKNAREITQDTNDITYADLNLPKGKKPA
PQAAEPNNHTEYASIQTSPQPASEDTLTYADLDMVHLNRTPKQPAPKPE
PSFSEYASVQVPRK.

In some embodiments, the SIRP-α peptide sequence comprises or consists of the wild type human SIRP-α sequence variant 4; NP_001317657; SEQ ID NO: 4. D1 domain in bold.

(SEQ ID NO: 4)
MEPAGPAPGRLGPLLCLLLAASCAWSGVAGEEELQVIQPDKSVLVAAGE
TATLRCTATSLIPVGPIQWFRGAGPGRELIYNQKEGHFPRVTTVSDLTK
RNNMDFSIRIGNITPADAGTYYCVKFRKGSPDDVEFKSGAGTELSVRAK
PSAPVVSGPAARATPQHTVSFTCESHGFSPRDITLKWFKNGNELSDFQT
NVDPVGESVSYSIHSTAKVVLTREDVHSQVICEVAHVTLQGDPLRGTAN
LSETIRVPPTLEVTQQPVRAENQVNVTCQVRKFYPQRLQLTWLENGNVS
RTETASTVTENKDGTYNWMSWLLVNVSAHRDDVKLTCQVEHDGQPAVSK
SHDLKVSAHPKEQGSNTAAENTGSNERNIYIVVGVVCTLLVALLMAALY
LVRIRQKKAQGSTSSTRLHEPEKNAREITQVQSLDTNDITYADLNLPKG
KKPAPQAAEPNNHTEYASIQTSPQPASEDTLTYADLDMVHLNRTPKQPA
PKPEPSFSEYASVQVPRK.

In some embodiments, the SIRP-α peptide sequence comprises or consists of the wild-type d1 domain of SIRP-α, SEQ ID NO: 5:

(SEQ ID NO: 5)
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPS.

In some embodiments, the SIRP-α peptide sequence comprises or consists of the mutant d1 domain of SIRP-α, SEQ ID NO: 6:

(SEQ ID NO: 6)
EEELQIIQPDKSVLVAAGETATLRCTITSLFPVGPIQWFRGAGPGRVLI
YNQRQGPFPRVTTVSDTTKRNNMDFSIRIGNITPADAGTYYCIKFRKGS
PDDVEFKSGAGTELSVRAKPS.

In some embodiments, the SIRP-α peptide sequence comprises or consists of the mutant d1 domain of SIRP-α, SEQ ID NO: 7, wherein X=any amino acid:

(SEQ ID NO: 7)
XXELQVIQPDKSVLVAAGETATLRCTATSLIPVGPIQWFRGAGPGRELI
YNQKEGHFPRVTTVSDLTKRNNMDFSIRIGNITPADAGTYYCVKFRKGS
PDDVEFKSGAGTELSVR.

In some embodiments, the SIRP-α peptide sequence comprises or consists of the mutant d1 domain of SIRP-α, SEQ ID NO: 8, wherein X=any amino acid:

(SEQ ID NO: 8)
XXELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVR.

In some embodiments, the SIRP-α peptide sequence comprises or consists of the mutant d1 domain of SIRP-α, SEQ ID NO: 9, wherein X=any amino acid:

(SEQ ID NO: 9)
EEXLQVIQPDKXVXVAAGEXAXLXCTXTSLIPVGPIQWFRGAGPXRELI
YNQKEGHFPRVTTVSXXDLTKRXNMDFXIXIXNITPADAGTYYCVKFRK
GSPDDXEFKSGAGTELSVR.

In some embodiments, the SIRP-α peptide sequence is monomeric. In some embodiments, the SIRP-α peptide sequence is multimeric. In some embodiments, the SIRP-α peptide sequence is fused to a human IgG constant region (Fc) sequence.

In some embodiments, the SIRP-α peptide sequence is fused to an IgG1 sequence and comprises or consists of SEQ ID NO: 10; wild type d1 domain (SEQ ID NO: 5) in bold

(SEQ ID NO: 10)
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPSDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GK .

In some embodiments, the SIRP-α peptide sequence is fused to an IgG4 sequence and comprises or consists of SEQ ID NO: 11; wild type d1 domain (SEQ ID NO: 5) in bold

(SEQ ID NO: 11)
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPSESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK

In some embodiments, the SIRP-α peptide sequence comprises one or more of the following mutations relative to SEQ ID NOS: 5-11: E3G, L4V, L4I, V6I, V6L, S12F, S14L, S20T, A21V, I22T, H24L, H24R, V27A, V27I, V27L, I31F, I31S, I31T, Q37H, A45G, E47V, E47L, K53R, E54Q, E54P, H56P, H56R, V631, E65D, S66T, S66G, S66L, K68R, E70N, M72R, S75P, R77S, S79G, N80A, N80X, I81N, T82N, P83N, P83X, V92I, F94L, F94V, duplication of D100, E102V, E102T, E102F, F103E, F103V, K104F, K104V, A115G, K116A, and/or K116G, wherein X=any amino acid.

In some embodiments, a SIRP-α peptide sequence may comprise or consist of any of the SIRP-α sequences described in WO2013109752, WO2014094122A1, WO2017027422, WO2016023040, and WO2016024021A1, incorporated herein in their entirety.

In some embodiments, a SIRP-α peptide sequence may comprise or consist of an amino acid sequence at least 50%, 55%, 60%, 65%, 70%, 75%, 80%, 85%, 90%, 91%, 92%, 93%, 94%, 95%, 96%, 97%, 98%, 99% or more homologous to any of SEQ ID NOS: 5-17, as shown in Table 3.

In some embodiments, high affinity SIRPα reagents is a CV1-hIgG4 or a CV1 monomer. In some embodiments, the dl domain of CV1-hIgG4 or CV1 monomer comprises the amino acid sequence as follows: (SEQ ID NO:12)

EEELQIIQPDKSVLVAAGETATLRCTITSLFPVGPIQWFRGAGPGRVLI
YNQRQGPFPRVTTVSDTTKRNNMDFSIRIGNITPADAGTYYCIKFRKGS
PDDVEFKSGAGTELSVRAKPS.

In some embodiments, the dl domain of CV1 is fused to an Fc domain. In some embodiments, when the dl domain of CV1 is fused to the human IgG4 Fc domain (i.e. CV1-hIgG4) it may comprise the amino acid sequence as follows: (SEQ ID NO: 13)

EEELQIIQPDKSVLVAAGETATLRCTITSLFPVGPIQWFRGAGPGRVLI
YNQRQGPFPRVTTVSDTTKRNNMDFSIRIGNITPADAGTYYCIKFRKGS
PDDVEFKSGAGTELSVRAKPSAAAPPCPPCPAPEFLGGPSVFLFPPKPK
DTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLS
PGK.

In some embodiments, when the dl domain of CV1 is fused to the human IgG2 Fc domain (i.e. CV1-hIgG2) it may comprise the amino acid sequence as follows: (SEQ ID NO: 14)

EEELQIIQPDKSVLVAAGETATLRCTITSLFPVGPIQWFRGAGPGRVLI
YNQRQGPFPRVTTVSDTTKRNNMDFSIRIGNITPADAGTYYCIKFRKGS
PDDVEFKSGAGTELSVRAKPSAAAVECPPCPAPPVAGPSVFLFPPKPKD
TLMISRTPEVTCVVVDVSHEDPEVQFNWYVDGMEVHNAKTKPREEQFNS
TFRVVSVLTVVHQDWLNGKEYKCKVSNKGLPAPIEKTISKTKGQPREPQ
VYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSP
GK.

In some embodiments, high affinity SIRPα reagents may be a FD6-hIgG4 or a FD6 monomer. In some embodiments, the dl domain of FD6-hIgG4 or a FD6 monomer comprises the amino acid sequence as follows: (SEQ ID NO:15)

EEEVQIIQPDKSVSVAAGESAILHCTITSLFPVGPIQWFRGAGPARVLI
YNQRQGPFPRVTTISETTRRENMDFSISISNITPADAGTYYCIKFRKGS
PDTEFKSGAGTELSVRAKPS.

In some embodiments, the dl domain of FD6 may be fused to an Fc domain. In some embodiments, when the dl domain of FD6 is fused to the human IgG4 Fc domain (i.e. FD6-hIgG4) it may comprise the amino acid sequence as follows: (SEQ ID NO: 16)

EEEVQIIQPDKSVSVAAGESAILHCTITSLFPVGPIQWFRGAGPARVLI
YNQRQGPFPRVTTISETTRRENMDFSISISNITPADAGTYYCIKFRKGS
PDTEFKSGAGTELSVRAKPSAAAPPCPPCPAPEFLGGPSVFLFPPKPKD
TLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSP
GK.

In some embodiments, when the dl domain of FD6 is fused to the human IgG2 Fc domain (i.e. FD6-hIgG2) it may comprise the amino acid sequence as follows: (SEQ ID NO: 17)

EEEVQIIQPDKSVSVAAGESAILHCTITSLFPVGPIQWFRGAGPARVLI
YNQRQGPFPRVTTISETTRRENMDFSISISNITPADAGTYYCIKFRKGS
PDTEFKSGAGTELSVRAKPSAAAVECPPCPAPPVAGPSVFLFPPKPKDT
LMISRTPEVTCVVVDVSHEDPEVQFNWYVDGMEVHNAKTKPREEQFNST
FRVVSVLTVVHQDWLNGKEYKCKVSNKGLPAPIEKTISKTKGQPREPQV
YTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPG
K.

Optionally the SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

In some embodiments, the therapeutic dosage may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly.

In some embodiments, the dosage of a SIRPα reagent for use in treating vascular inflammation is from about 0.0001 to 100 mg/kg of the host body weight. In some embodiments, the dosage of a SIRPα reagent for use in treating vascular inflammation is from about 0.01 to 5 mg/kg of the host body weight. For example, dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly intervals.

The term “therapeutically effective dose” refers to a dose that produces the effects for which it is administered. The exact dose will depend on the purpose of the treatment. In some embodiments, adjustments for polypeptide construct degradation, systemic versus localized delivery, and rate of new protease synthesis, as well as the age, body weight, general health, sex, diet, time of administration, drug interaction and the severity of the condition is necessary.

A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%. The variant polypeptides can be naturally or non-naturally glycosylated, i.e., the polypeptide has a glycosylation pattern that differs from the glycosylation pattern found in the corresponding naturally occurring protein. The variant polypeptides can have post-translational modifications not found on the natural protein.

A “fusion” polypeptide is a polypeptide comprising a polypeptide or portion (e.g., one or more domains) thereof fused or bonded to heterologous polypeptide. A fusion soluble protein, for example, will share at least one biological property in common with a native sequence soluble polypeptide. Examples of fusion polypeptides include immunoadhesins, as described above, which combine a portion of the polypeptide of interest with an immunoglobulin sequence, and epitope tagged polypeptides, which comprise a soluble polypeptide of interest or portion thereof fused to a “tag polypeptide”. The tag polypeptide has enough residues to provide an epitope against which an antibody can be made, yet is short enough such that it does not interfere with biological activity of the polypeptide of interest. Suitable tag polypeptides generally have at least six amino acid residues and usually between about 6-60 amino acid residues.

Anti-CD47 antibodies. In some embodiments, a subject anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Some anti-CD47 antibodies do not reduce the binding of CD47 to SIRPα (and are therefore not considered to be an “anti-CD47 agent” herein) and such an antibody can be referred to as a “non-blocking anti-CD47 antibody”. A suitable anti-CD47 antibody that is an “anti-CD47 agent” can be referred to as a “CD47-blocking antibody”. Non-limiting examples of suitable antibodies include lemzoparlimab, STI-6643; IMC-002; CC-90002 (Celgene), SRF231 (Surface Oncology), SHR-1603 (Hengrui), and IB1188 (Innovent Biologics). B6H12, 5F9 (magrolimab), 8B6, and C3 are described in U.S. Pat. No. 9,017,675, herein specifically incorporated by reference. An antibody may bind to the epitope recognized by magrolimab. Suitable anti-CD47 antibodies include fully human, humanized or chimeric versions of such antibodies. Humanized antibodies are especially useful for in vivo applications in humans due to their low antigenicity.

In some embodiments an anti-CD47 antibody comprises a human IgG Fc region, e.g. an IgG1, IgG2a, IgG2b, IgG3, IgG4 constant region. In a preferred embodiment the IgG Fc region is an IgG4 constant region. The IgG4 hinge may be stabilized by the amino acid substitution S241P (see Angal et al. (1993) Mol. Immunol. 30(1):105-108, herein specifically incorporated by reference).

Anti-SIRPα antibodies. In some embodiments, a subject anti-CD47 agent is an antibody that specifically binds SIRPα (i.e., an anti-SIRPα antibody) and reduces the interaction between CD47 on one cell (e.g., an infected cell) and SIRPα on another cell (e.g., a phagocytic cell). Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Statins are inhibitors of HMG-CoA reductase enzyme. These agents are described in detail in various publications. For example, mevastatin and related compounds are disclosed in U.S. Pat. No. 3,983,140, lovastatin (mevinolin) and related compounds are disclosed in U.S. Pat. No. 4,231,938, pravastatin and related compounds are disclosed in U.S. Pat. No. 4,346,227, simvastatin and related compounds are disclosed in U.S. Pat. Nos. 4,448,784 and 4,450,171; fluvastatin and related compounds are disclosed in U.S. Pat. No. 5,354,772; atorvastatin and related compounds are disclosed in U.S. Pat. Nos. 4,681,893, 5,273,995 and 5,969,156; and cerivastatin and related compounds are disclosed in U.S. Pat. Nos. 5,006,530 and 5,177,080; rosuvastatin and related compounds are disclosed in European Patent Application Publication No. 0521471 and U.S. Pat. No. 6,858,618; pitavastatin and related compounds are disclosed in U.S. Pat. No. 5,856,336. Additional statin compounds are disclosed in U.S. Pat. Nos. 5,208,258, 5,130,306, 5,116,870, 5,049,696, RE 36,481, and RE 36,520. Statins include the salts and/or ester thereof.

For the purposes of the present invention, an effective dose of a statin in a combination with anti-CD47 agent (or salt or ester thereof) is the dose that, when administered for a suitable period of time, usually at least about one week, about two weeks or more, or up to extended periods of time such as months or years, will evidence a reduction in the progression of the disease, e.g. vascular inflammation, atherosclerosis, and the like. It will be understood by those of skill in the art that an initial dose may be administered for such periods of time, followed by maintenance doses, which, in some cases, will be at a reduced dosage.

The formulation and administration of statins is well known, and will generally follow conventional usage. The dosage required to treat inflammation may be commensurate with the dose used in the treatment of high cholesterol. In some embodiments, the dose of the statin used to treat vascular inflammation is reduced relative to a standard dose. For example, lovastatin may be administered in a daily dose of at least about 1 mg, at least about 5 mg, at least about 10 mg, and not more than about 250 mg, not more than about 150 mg, or not more than about 80 mg, inclusive of a values, ranges, and subranges therebetween. The use of statins in general and lovastatin in particular can be at doses from about 1-250 mg (about 0.01-2.5 mg/kg). Specific examples of statins useful in the methods of the invention are atorvastatin (LIPITOR™); cerivastatin (LIPOBAY™); fluvastatin (LESCOL™); lovastatin (MEVACOR™); mevastatin (COMPACTIN™); pitavastatin (LIVALO™); pravastatin (PRAVACHOL™); Rosuvastatin (CRESTOR™); simvastatin (ZOCOR™); etc.

The use of combination therapy may allow lower doses of each monotherapy than currently used in standard practice while achieving significant efficacy, including efficacy beyond that conventional dosing of either monotherapy. Those of skill in the art will readily appreciate that dose levels can vary as a function of the specific compound, the severity of the symptoms, and the susceptibility of the subject to side effects. Some of the specific compounds are more potent than others. Preferred dosages for a given compound are readily determinable by those of skill in the art by a variety of means. A preferred means is to measure the physiological potency of a given compound. The use of combination therapy may allow lower doses of each monotherapy than currently used in standard practice while achieving significant efficacy, including efficacy greater than that achieved by conventional dosing of either monotherapy. In particular embodiments the combination provides for a synergistic improvement in disease markers or disease symptoms over the administration of either drug as a single agent.

In some embodiments the statin dose is reduced relative to the conventional dose, e.g. reduced up to about 10%, up to about 20%, up to about 30%, up to about 40%, up to about 50%, up to about 60%, 70% or more, relative to a monotherapy dose. In some embodiments a dose of an anti-CD47 agent is reduced up to about 10%, up to about 20%, up to about 30%, up to about 40%, up to about 50%, up to about 60%, 70% or more, relative to a monotherapy dose. In some embodiments the dose of both statin and anti-CD47 agent are reduced, each by up to about 10%, up to about 20%, up to about 30%, up to about 40%, up to about 50%, up to about 60%, 70% or more, relative to a monotherapy dose.

For demonstrating additive or synergistic activity of the two drugs (e.g., anti-CD47 agent and a statin such as lovastatin) and establishing an appropriate dose ratio for clinical investigation, varying amounts of the two drugs are administered to appropriate clinical or pre-clinical models of vascular inflammatory disease. Alternatively, the effects of varying amounts of the two drugs are tested on a cellular response mediating inflammation that may be involved in the pathogenesis of disease.

It is within the level of skill of a clinician to determine the preferred route of administration and the corresponding dosage form and amount, as well as the dosing regimen, i.e., the frequency of dosing. In particular embodiments, the combination therapy will be delivered in once-a-day (s.i.d.) dosing. In other embodiments, twice-a-day (b.i.d.) dosing may be used. However, this generalization does not take into account such important variables as the specific type of inflammatory disease, the specific therapeutic agent involved and its pharmacokinetic profile, and the specific individual involved. For an approved product in the marketplace, much of this information is already provided by the results of clinical studies carried out to obtain such approval. In other cases, such information may be obtained in a straightforward manner in accordance with the teachings and guidelines contained in the instant specification taken in light of the knowledge and skill of the artisan. The results that are obtained can also be correlated with data from corresponding evaluations of an approved product in the same assays.

9p21 Risk. As used herein, the term “an individual carrying at least one 9p21 risk factor” refers to humans in which one or more risk alleles at the 9p21 locus are present in the genome. Such individuals have been shown to have an increased risk of: early onset myocardial infarction, abominal aortic aneurysm, stroke, peripheral artery disease, and myocardial infarction/coronary heart disease. This risk is independent of traditional risk factors, including diabetes, hypertension, cholesterol, and obesity. See, for example, Helgadottir et al. Science. 2007; 316(5830):1491-1493; Helgadottir et al. Nat Genet. 2008; 40(2):217-224; Palomaki et al. JAMA. 2010; 303(7):648-656; and Roberts et al. Curr Opin Cardiol. 2008; 23:629-633, each herein specifically incorporated by reference.

The 9p21 locus is in tight LD (linkage disequilibrium), and a number of single nucleotide polymorphisms (SNP) markers have been shown to be useful in diagnosis. Representative SNPs include without limitation rs10757278; rs3217992; rs4977574; rs1333049; rs10757274; rs2383206; rs2383207; Rs3217989; rs1333040; rs2383207; rs10116277; rs7044859; rs1292136; rs7865618; rs1333045; rs9632884; rs10757272; rs4977574; rs2891168; rs6475606; rs1333048; rs1333049; Rs1333045; etc. In some embodiments an individual is treated without genotypic analysis of the locus.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′).sub.2, Fab, Fv and rlgG. The term also refers to recombinant single chain Fv fragments (scFv). The term antibody also includes bivalent or bispecific molecules, diabodies, triabodies, and tetrabodies.

Selection of antibodies may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies.

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Thus pepsin digests an antibody below the disulfide linkages in the hinge region to produce F(ab)′.sub.2, a dimer of Fab which itself is a light chain joined to V.sub.H-C.sub.H1 by a disulfide bond. The F(ab)′.sub.2 may be reduced under mild conditions to break the disulfide linkage in the hinge region, thereby converting the F(ab)′.sub.2 dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Antibodies of interest may be tested for their ability to induce ADCC (antibody-dependent cellular cytotoxicity) or ADCP (antibody dependent cellular phagocytosis). Antibody-associated ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-11-dUTP nick end labeling (TUNEL) assay (Lazebnik et al., Nature: 371, 346 (1994). Cytotoxicity may also be detected directly by detection kits known in the art, such as Cytotoxicity Detection Kit from Roche Applied Science (Indianapolis, Ind.).

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The term “diagnosis” is used herein to refer to the identification of a molecular or pathological state, disease or condition, such as the identification of a molecular subtype of cardiovascular disease.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of of cardiovascular disease, e.g. reduction of inflammation in a human, and includes inhibiting the disease, i.e., arresting its development; and relieving the disease, i.e., causing regression of the disease.

Treating may refer to any indicia of success in the treatment or amelioration or prevention of disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect. Typically a regular (daily) dosing of a statin will be maintained, with one or more doses of an anti-CD47 agent.

“Concomitant administration” of a statin with an anti-CD47 agent means administration at such time that both will have a therapeutic effect. Such concomitant administration may involve concurrent (i.e. at the same time), prior, or subsequent administration. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C.sub.1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

The terms “phagocytic cells” and “phagocytes” are used interchangeably herein to refer to a cell that is capable of phagocytosis. There are three main categories of phagocytes: macrophages, mononuclear cells (histiocytes and monocytes); polymorphonuclear leukocytes (neutrophils) and dendritic cells. However, “non-professional” cells are also known to participate in efferocytosis, such as neighboring SMCs in the blood vessel wall.

A “therapeutically effective amount” means the amount that, when administered to a subject for treating a disease, is sufficient to effect treatment for that disease. An “effective amount” can be an amount sufficient to effect beneficial or desired clinical results. An effective amount can be administered in one or more administrations. For purposes of this invention, an effective amount of an anti-CD47 agent is an amount that is sufficient to palliate, ameliorate, stabilize, reverse, prevent, slow or delay the progression of the disease state, e.g. atherosclerosis or atherosclerotic plaque, by increasing phagocytosis of a target cell. For example, in an animal model the percent of aortic surface area with atherosclerotic plaque may be reduced 25%, 50%, 75% or more relative to a control treated animal. Similar effects may be obtained with indicia appropriate for human patients, including without limitation C-reactive protein [CRP] and fibrinogen; lipoprotein-associated phospholipase A2 [Lp-PLA2] and myeloperoxidase [MPO]; growth differentiation factor-15 [GDF-15]) inflammatory markers; ambulatory arterial stiffness, IVUS imaging, and the like. See, for example Krintus et al. (2013) Crit Rev Clin Lab Sci. 11:1-17; Kollias et al. (2012) Atherosclerosis 224(2):291-301; and Kollias et al. (2011) Int. J. Cardiovasc. Imaging 27(2):225-37, each herein specifically incorporated by reference.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations. The definition also includes sample that have been enriched for particular types of molecules, e.g., nucleic acids, polypeptides, etc.

A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. The term “derivative” encompasses both amino acid sequence variants of polypeptide and covalent modifications thereof. For example, derivatives and fusion of soluble CRT find use as CRT mimetic molecules.

Efferocytosis. The process by which professional and nonprofessional phagocytes dispose of apoptotic cells in a rapid and efficient manner. Efferocytosis involves a number of molecules, including ligands on the apoptotic cells, e.g. phosphatidylserine; receptors on the efferocyte; soluble ligand-receptor bridging molecules; and so-called “find-me” and “don't-eat me” molecules, (e.g., lysosphospholipids and CD47, respectively) the expression of which by dying cells is altered to attract nearby phagocytes. By clearing apoptotic cells at a relatively early stage of cell death, when the cell plasma and organelle membranes are still intact, postapoptotic, or “secondary”, necrosis is prevented. Prevention of cellular necrosis, in turn, prevents the release of potentially damaging intracellular molecules into the extracellular milieu, including molecules that can stimulate inflammatory, proatherosclerotic and/or autoimmune responses.

The efficiency of efferocytic clearance in atherosclerotic lesions plays a key role in disease development. Efferocytosis is known to be impaired in human atherosclerotic plaque. A prominent feature of advanced atherosclerotic lesions is the necrotic core, or lipid core, which is a collection of dead and necrotic macrophages surrounded by inflammatory cells. Necrotic cores are thought to be a major feature responsible for plaque “vulnerability”, i.e., plaques capable of undergoing disruption and triggering acute lumenal thrombosis. Plaque disruption and acute thrombosis are the events that trigger acute coronary syndromes, including myocardial infarction, unstable angina, sudden cardiac death, and stroke.

### Methods

The anti-CD47 agents of the disclosure are particularly effective in treating or reducing vascular inflammation in a subject. In this regard, it will be appreciated that the anti-CD47 agents of the disclosure, including anti-CD47 antibodies of the disclosure, are used to treat, reduce, control, suppress, modulate, or eliminate unwanted vascular inflammation in a subject. In some embodiments, the anti-CD47 agents of the disclosure are used to treat vascular inflammation in a subject. In some embodiments, the anti-CD47 agents of the disclosure are used to reduce vascular inflammation in a subject. In some embodiments, vascular inflammation is cardiovascular inflammation.

In some aspects, the anti-CD47 agents of the disclosure are useful to treat vascular inflammation in a human subject by administering the anti-CD47 agent of the disclosure in an effective amount to the human subject in need thereof, thereby treating vascular inflammation. Any route of administration suitable for achieving the desired effect is contemplated by the disclosure (e.g., intravenous, intramuscular, subcutaneous). Treatment or reduction of vascular inflammation may result in a decrease in the symptoms associated with the condition, which may be long-term or short-term, or even transient beneficial effect.

In some embodiments, the anti-CD47 agents of the disclosure are administered to subjects in need thereof to treat vascular inflammation. In some embodiments, the anti-CD47 agents of the disclosure are administered to subjects in need thereof to reduce vascular inflammation.

As used herein, the terms “marker of inflammation” and “indicia of inflammation” may be used interchangeably. In some embodiments a “marker of inflammation” or “indicia of inflammation” is an indicator of inflammation. In some embodiments, the level of a marker of inflammation or indicia of inflammation can be assayed. In some embodiments, a subject with inflammation (for example, vascular inflammation), may have increased levels of a marker of inflammation. In some embodiments, a subject with inflammation (for example, vascular inflammation), may have altered levels of a marker of inflammation. In some embodiments, an anti-CD47 agent of the disclosure is used to treat or reduce inflammation in a subject. In some embodiments, an anti-CD47 agent of the disclosure is used to treat or reduce inflammation in a subject and thereby reduced the levels of a marker of inflammation in the subject.

In some aspects, the effectiveness of an anti-CD47 agent of the disclosure is demonstrated by comparing the levels of a marker of inflammation in human subjects treated with an anti-CD47 agent of the disclosure to the levels of a marker of inflammation in a human subject treated with placebo or a control formulation. In some aspects, the effectiveness of an anti-CD47 agent of the disclosure is demonstrated by comparing the levels of a marker of inflammation in human subjects prior to treatment with an anti-CD47 agent of the disclosure and following treatment with an anti-CD47 agent of the disclosure. In some embodiments, the levels of a marker of inflammation are reduced in a human subject following treatment with an anti-CD47 agent of the disclosure. In some embodiments, the levels of a marker of inflammation are altered in a human subject following treatment with an anti-CD47 agent of the disclosure.

In some embodiments, the level of a marker of inflammation in a human subject is reduced by at least about 5%, 10%, 15%, 20%, 25%, 30%, 35%, 40%, 45%, 50% or more following treatment with an anti-CD47 agent of the disclosure. In some embodiments, the level of a marker of inflammation in a human subject is reduced by at least about 5%, 10%, 15%, 20%, 25%, 30%, 35%, 40%, 45%, 50% or more following treatment with an anti-CD47 agent of the disclosure when compared to the levels of the marker of inflammation in the human subject prior to treatment with the anti-CD47 agent. In some embodiments, the level of a marker of inflammation in a human subject is reduced by at least about 5%, 10%, 15%, 20%, 25%, 30%, 35%, 40%, 45%, 50% or more following treatment with an anti-CD47 agent of the disclosure when compared to the levels of the marker of inflammation in a human subject treated with placebo or a control formulation. In some embodiments, the level of a marker of inflammation in a human subject is reduced by at least about 5% or more following treatment with an anti-CD47 agent of the disclosure. In some embodiments, the level of a marker of inflammation in a human subject is reduced by at least about 10% or more following treatment with an anti-CD47 agent of the disclosure. In some embodiments, the level of a marker of inflammation in a human subject is reduced by at least about 15% or more following treatment with an anti-CD47 agent of the disclosure. In some embodiments, the level of a marker of inflammation in a human subject is reduced by at least about 20% or more following treatment with an anti-CD47 agent of the disclosure. In some embodiments, the level of a marker of inflammation in a human subject is reduced by at least about 25% or more following treatment with an anti-CD47 agent of the disclosure. In some embodiments, the level of a marker of inflammation in a human subject is reduced by at least about 30% or more following treatment with an anti-CD47 agent of the disclosure. In some embodiments, the level of a marker of inflammation in a human subject is reduced by at least about 35% or more following treatment with an anti-CD47 agent of the disclosure. In some embodiments, the level of a marker of inflammation in a human subject is reduced by at least about 40% or more following treatment with an anti-CD47 agent of the disclosure. In some embodiments, the level of a marker of inflammation in a human subject is reduced by at least about 45% or more following treatment with an anti-CD47 agent of the disclosure. In some embodiments, the level of a marker of inflammation in a human subject is reduced by at least about 50% or more following treatment with an anti-CD47 agent of the disclosure.

The levels of a marker of inflammation in a human subject (for example, a sample from a subject with vascular inflammation) are assayed by conventional methods known to those of skill in the art.

In some embodiments, a marker of inflammation is selected from ¹⁸F-FDG uptake as measured by positron emission tomography (PET) performed with fluorine 18 fluorodeoxyglucose (FDG) combined with computed tomography (CT), high sensitivity C-reactive protein (hsCRP), C-reactive protein (CRP), IL-6, IL-8, fibrinogen, Human serum amyloid A (SAA), Haptoglobin (Hp), secretory phospholipase A2 (sPLA2), Lipoprotein(a), apolipoprotein B (APOB) to apolipoprotein A1 (APOA1) ratio, and white blood cell count (WBC). (Ridker et al., Lancet 2021 May 29; 397(10289):2060-2069; Ridker et al., Lancet 2018 Jan. 27; 391(10118):319-328).

In some embodiments, a marker of inflammation is ¹⁸F-FDG uptake as measured by positron emission tomography (PET) performed with fluorine 18 fluorodeoxyglucose (FDG) combined with computed tomography (CT) (hereafter PET/CT) In some embodiments, the effectiveness of an anti-CD47 agent of the disclosure is measured by comparing ¹⁸F-FDG uptake in a human subject treated with an anti-CD47 agent of the disclosure to ¹⁸F-FDG uptake in a human subject treated with placebo or a control formulation. In some embodiments, the effectiveness of an anti-CD47 agent of the disclosure is measured by comparing ¹⁸F-FDG uptake in a human subject prior to treatment with an anti-CD47 agent of the disclosure and following treatment with an anti-CD47 agent of the disclosure. In some embodiments, after treatment with an anti-CD47 agent of the disclosure, ¹⁸F-FDG uptake in the subject are reduced when compared to ¹⁸F-FDG uptake in the patient prior to treatment and/or when compared to a human subject treated with placebo or a control formulation. In some embodiments, after treatment with an anti-CD47 agent of the disclosure, ¹⁸F-FDG uptake in the subject are altered when compared to ¹⁸F-FDG uptake in the patient prior to treatment and/or when compared to a human subject treated with placebo or a control formulation.

In some embodiments, ¹⁸F-FDG uptake is expressed as the target-to-background ratio (TBR). In some embodiments, after treatment with an anti-CD47 agent of the disclosure, the TBR of ¹⁸F-FDG uptake in the subject is reduced when compared to the TBR of ¹⁸F-FDG uptake in the patient prior to treatment and/or when compared to a human subject treated with placebo or a control formulation. In some embodiments, after treatment with an anti-CD47 agent of the disclosure, the TBR of ¹⁸F-FDG uptake in the subject is altered when compared to the TBR of ¹⁸F-FDG uptake in the patient prior to treatment and/or when compared to a human subject treated with placebo or a control formulation.

In some embodiments, ¹⁸F-FDG uptake is measured as maximum standardized uptake values (SUV). In some embodiments, after treatment with an anti-CD47 agent of the disclosure, the maximum SUV of ¹⁸F-FDG uptake in the subject is reduced when compared to the maximum SUV of ¹⁸F-FDG uptake in the patient prior to treatment and/or when compared to a human subject treated with placebo or a control formulation. In some embodiments, after treatment with an anti-CD47 agent of the disclosure, the maximum SUV of ¹⁸F-FDG uptake in the subject is altered when compared to the maximum SUV of ¹⁸F-FDG uptake in the patient prior to treatment and/or when compared to a human subject treated with placebo or a control formulation.

In some embodiments, a marker of inflammation is high sensitivity C-reactive protein (hsCRP). In some embodiments, the effectiveness of an anti-CD47 agent of the disclosure is measured by comparing the levels of high sensitivity C-reactive protein (hsCRP) in a human subject treated with an anti-CD47 agent of the disclosure to the levels of hsCRP in a human subject treated with placebo or a control formulation. In some embodiments, the effectiveness of an anti-CD47 agent of the disclosure is measured by comparing the levels of high sensitivity C-reactive protein (hsCRP) in a human subject prior to treatment with an anti-CD47 agent of the disclosure and following treatment with an anti-CD47 agent of the disclosure. In some embodiments, after treatment with an anti-CD47 agent of the disclosure, levels of high sensitivity C-reactive protein (hsCRP) in the subject are reduced when compared to the level of high sensitivity C-reactive protein (hsCRP) in the patient prior to treatment and/or when compared to a human subject treated with placebo or a control formulation. In some embodiments, after treatment with an anti-CD47 agent of the disclosure, levels of high sensitivity C-reactive protein (hsCRP) in the subject are altered when compared to the level of high sensitivity C-reactive protein (hsCRP) in the patient prior to treatment and/or when compared to a human subject treated with placebo or a control formulation.

In some embodiments, a marker of inflammation is C-reactive protein (CRP). In some embodiments, the effectiveness of an anti-CD47 agent of the disclosure is measured by comparing the levels of CRP in a human subject treated with an anti-CD47 agent of the disclosure to the levels of CRP in a human subject treated with placebo or a control formulation. In some embodiments, the effectiveness of an anti-CD47 agent of the disclosure is measured by comparing the levels of CRP in a human subject prior to treatment with an anti-CD47 agent of the disclosure and following treatment with an anti-CD47 agent of the disclosure. In some embodiments, after treatment with an anti-CD47 agent of the disclosure, levels of CRP in the subject are reduced when compared to the level of CRP in the patient prior to treatment and/or when compared to a human subject treated with placebo or a control formulation. In some embodiments, after treatment with an anti-CD47 agent of the disclosure, levels of CRP in the subject are altered when compared to the level of CRP in the patient prior to treatment and/or when compared to a human subject treated with placebo or a control formulation.

In some embodiments, a marker of inflammation is IL-6. In some embodiments, the effectiveness of an anti-CD47 agent of the disclosure is measured by comparing the levels of IL-6 in a human subject treated with an anti-CD47 agent of the disclosure to the levels of IL-6 in a human subject treated with placebo or a control formulation. In some embodiments, the effectiveness of an anti-CD47 agent of the disclosure is measured by comparing the levels of IL-6 in a human subject prior to treatment with an anti-CD47 agent of the disclosure and following treatment with an anti-CD47 agent of the disclosure. In some embodiments, after treatment with an anti-CD47 agent of the disclosure, levels of IL-6 in the subject are reduced when compared to the level of IL-6 in the patient prior to treatment and/or when compared to a human subject treated with placebo or a control formulation. In some embodiments, after treatment with an anti-CD47 agent of the disclosure, levels of IL-6 in the subject are altered when compared to the level of IL-6 in the patient prior to treatment and/or when compared to a human subject treated with placebo or a control formulation.

In some embodiments, a marker of inflammation is IL-8. In some embodiments, the effectiveness of an anti-CD47 agent of the disclosure is measured by comparing the levels of IL-8 in a human subject treated with an anti-CD47 agent of the disclosure to the levels of IL-8 in a human subject treated with placebo or a control formulation. In some embodiments, the effectiveness of an anti-CD47 agent of the disclosure is measured by comparing the levels of IL-8 in a human subject prior to treatment with an anti-CD47 agent of the disclosure and following treatment with an anti-CD47 agent of the disclosure. In some embodiments, after treatment with an anti-CD47 agent of the disclosure, levels of IL-8 in the subject are reduced when compared to the level of IL-8 in the patient prior to treatment and/or when compared to a human subject treated with placebo or a control formulation. In some embodiments, after treatment with an anti-CD47 agent of the disclosure, levels of IL-8 in the subject are altered when compared to the level of IL-8 in the patient prior to treatment and/or when compared to a human subject treated with placebo or a control formulation.

In some embodiments, a marker of inflammation is fibrinogen. In some embodiments, the effectiveness of an anti-CD47 agent of the disclosure is measured by comparing the levels of fibrinogen in a human subject treated with an anti-CD47 agent of the disclosure to the levels of fibrinogen in a human subject treated with placebo or a control formulation. In some embodiments, the effectiveness of an anti-CD47 agent of the disclosure is measured by comparing the levels of fibrinogen in a human subject prior to treatment with an anti-CD47 agent of the disclosure and following treatment with an anti-CD47 agent of the disclosure. In some embodiments, after treatment with an anti-CD47 agent of the disclosure, levels of fibrinogen in the subject are reduced when compared to the level of fibrinogen in the patient prior to treatment and/or when compared to a human subject treated with placebo or a control formulation. In some embodiments, after treatment with an anti-CD47 agent of the disclosure, levels of fibrinogen in the subject are altered when compared to the level of fibrinogen in the patient prior to treatment and/or when compared to a human subject treated with placebo or a control formulation.

In some embodiments, a marker of inflammation is Human serum amyloid A (SAA). In some embodiments, the effectiveness of an anti-CD47 agent of the disclosure is measured by comparing the levels of SAA in a human subject treated with an anti-CD47 agent of the disclosure to the levels of SAA in a human subject treated with placebo or a control formulation. In some embodiments, the effectiveness of an anti-CD47 agent of the disclosure is measured by comparing the levels of SAA in a human subject prior to treatment with an anti-CD47 agent of the disclosure and following treatment with an anti-CD47 agent of the disclosure. In some embodiments, after treatment with an anti-CD47 agent of the disclosure, levels of SAA in the subject are reduced when compared to the level of SAA in the patient prior to treatment and/or when compared to a human subject treated with placebo or a control formulation. In some embodiments, after treatment with an anti-CD47 agent of the disclosure, levels of SAA in the subject are altered when compared to the level of SAA in the patient prior to treatment and/or when compared to a human subject treated with placebo or a control formulation.

In some embodiments, a marker of inflammation is Haptoglobin (Hp). In some embodiments, the effectiveness of an anti-CD47 agent of the disclosure is measured by comparing the levels of Hp in a human subject treated with an anti-CD47 agent of the disclosure to the levels of Hp in a human subject treated with placebo or a control formulation. In some embodiments, the effectiveness of an anti-CD47 agent of the disclosure is measured by comparing the levels of Hp in a human subject prior to treatment with an anti-CD47 agent of the disclosure and following treatment with an anti-CD47 agent of the disclosure. In some embodiments, after treatment with an anti-CD47 agent of the disclosure, levels of Hp in the subject are reduced when compared to the level of Hp in the patient prior to treatment and/or when compared to a human subject treated with placebo or a control formulation. In some embodiments, after treatment with an anti-CD47 agent of the disclosure, levels of Hp in the subject are altered when compared to the level of Hp in the patient prior to treatment and/or when compared to a human subject treated with placebo or a control formulation.

In some embodiments, a marker of inflammation is secretory phospholipase A2 (sPLA2). In some embodiments, the effectiveness of an anti-CD47 agent of the disclosure is measured by comparing the levels of sPLA2 in a human subject treated with an anti-CD47 agent of the disclosure to the levels of sPLA2 in a human subject treated with placebo or a control formulation. In some embodiments, the effectiveness of an anti-CD47 agent of the disclosure is measured by comparing the levels of sPLA2 in a human subject prior to treatment with an anti-CD47 agent of the disclosure and following treatment with an anti-CD47 agent of the disclosure. In some embodiments, after treatment with an anti-CD47 agent of the disclosure, levels of sPLA2 in the subject are reduced when compared to the level of sPLA2 in the patient prior to treatment and/or when compared to a human subject treated with placebo or a control formulation. In some embodiments, after treatment with an anti-CD47 agent of the disclosure, levels of sPLA2 in the subject are altered when compared to the level of sPLA2 in the patient prior to treatment and/or when compared to a human subject treated with placebo or a control formulation.

In some embodiments, a marker of inflammation is lipoprotein(a). In some embodiments, the effectiveness of an anti-CD47 agent of the disclosure is measured by comparing the levels of lipoprotein(a) in a human subject treated with an anti-CD47 agent of the disclosure to the levels of lipoprotein(a) in a human subject treated with placebo or a control formulation. In some embodiments, the effectiveness of an anti-CD47 agent of the disclosure is measured by comparing the levels of lipoprotein(a) in a human subject prior to treatment with an anti-CD47 agent of the disclosure and following treatment with an anti-CD47 agent of the disclosure. In some embodiments, after treatment with an anti-CD47 agent of the disclosure, levels of lipoprotein(a) in the subject are reduced when compared to the level of lipoprotein(a) in the patient prior to treatment and/or when compared to a human subject treated with placebo or a control formulation. In some embodiments, after treatment with an anti-CD47 agent of the disclosure, levels of lipoprotein(a) in the subject are altered when compared to the level of lipoprotein(a) in the patient prior to treatment and/or when compared to a human subject treated with placebo or a control formulation.

In some embodiments, a marker of inflammation is the apolipoprotein B (APOB) to apolipoprotein A1 (APOA1) ratio. In some embodiments, the effectiveness of an anti-CD47 agent of the disclosure is measured by comparing the levels of the apolipoprotein B (APOB) to apolipoprotein A1 (APOA1) ratio in a human subject treated with an anti-CD47 agent of the disclosure to the APOB to APOA1 ratio in a human subject treated with placebo or a control formulation. In some embodiments, the effectiveness of an anti-CD47 agent of the disclosure is measured by comparing the levels of the APOB to APOA1 ratio in a human subject prior to treatment with an anti-CD47 agent of the disclosure and following treatment with an anti-CD47 agent of the disclosure. In some embodiments, after treatment with an anti-CD47 agent of the disclosure, levels of the APOB to APOA1 ratio in the subject are reduced when compared to the level of the APOB to APOA1 ratio in the patient prior to treatment and/or when compared to a human subject treated with placebo or a control formulation. In some embodiments, after treatment with an anti-CD47 agent of the disclosure, levels of the APOB to APOA1 ratio in the subject are altered when compared to the level of the APOB to APOA1 ratio in the patient prior to treatment and/or when compared to a human subject treated with placebo or a control formulation.

In some embodiments, a marker of inflammation is white blood cell count (WBC). In some embodiments, the effectiveness of an anti-CD47 agent of the disclosure is measured by comparing the levels of WBC in a human subject treated with an anti-CD47 agent of the disclosure to the levels of WBC in a human subject treated with placebo or a control formulation. In some embodiments, the effectiveness of an anti-CD47 agent of the disclosure is measured by comparing the levels of WBC in a human subject prior to treatment with an anti-CD47 agent of the disclosure and following treatment with an anti-CD47 agent of the disclosure. In some embodiments, after treatment with an anti-CD47 agent of the disclosure, levels of WBC in the subject are reduced when compared to the level of WBC in the patient prior to treatment and/or when compared to a human subject treated with placebo or a control formulation. In some embodiments, after treatment with an anti-CD47 agent of the disclosure, levels of WBC in the subject are altered when compared to the level of WBC in the patient prior to treatment and/or when compared to a human subject treated with placebo or a control formulation.

The anti-CD47 agents of the disclosure and/or pharmaceutical compositions of the disclosure, are formulated into pharmaceutically acceptable dosage forms for human subjects by conventional methods known to those of skill in the art. In some embodiments, the actual dosage levels of the active ingredient (e.g., anti-CD47 agent) in the pharmaceutical compositions of the disclosure are varied so as to obtain an amount of the active ingredient which is effective to achieve the desired therapeutic response for a human subject, without being unacceptably toxic.

In some embodiments, a suitable dose of an anti-CD47 agent of the disclosure is an amount of the active ingredient which is the lowest dose effective to produce a therapeutic effect in a human subject. In some embodiments, dosages of the anti-CD47 agent of the disclosure or pharmaceutical composition of the disclosure range from approximately 20 mg/kg to 45 mg/kg of body weight per week.

In some embodiments, the anti-CD47 agent of the disclosure or pharmaceutical composition of the disclosure are administered in doses to humans from about 20 mg/kg to about 45 mg/kg per week. In some embodiments, dosages of greater than 45 mg/kg per week may be necessary.

In some embodiments, the anti-CD47 agent of the disclosure or pharmaceutical composition of the disclosure is administered to human patients, weekly, biweekly, monthly, or semi-monthly (for example, every two months or every three months) at a dose of about 20 mg/kg, 30 mg/kg, or 45 mg/kg.

In some embodiments, the anti-CD47 agent of the disclosure is administered to human patients weekly, for at least two weeks. In some embodiments, the anti-CD47 agent of the disclosure is administered to human patients weekly, for at least three weeks. In some embodiments, the anti-CD47 agent of the disclosure is administered to human patients weekly, for at least four weeks. In some embodiments, the anti-CD47 agent of the disclosure is administered to human patients weekly, for at least five weeks. In some embodiments, the anti-CD47 agent of the disclosure is administered to human patients weekly, for at least six weeks. In some embodiments, the anti-CD47 agent of the disclosure is administered to human patients weekly, for at least seven weeks. In some embodiments, the anti-CD47 agent of the disclosure is administered to human patients weekly, for at least eight weeks. In some embodiments, the anti-CD47 agent of the disclosure is administered to human patients weekly, for at least nine weeks.

In some embodiments, the anti-CD47 agent of the disclosure is administered to human patients monthly, for at least two months.

In some embodiments, the anti-CD47 agent of the disclosure is administered to a human patient at a dose of 20 mg/kg weekly. In some embodiments, the anti-CD47 agent of the disclosure is administered to a human patient at a dose of 30 mg/kg weekly. In some embodiments, the anti-CD47 agent of the disclosure is administered to a human patient at a dose of 45 mg/kg weekly.

In some embodiments, the anti-CD47 agent of the disclosure is administered to a human patient at a dose of 20 mg/kg weekly for at least nine weeks. In some embodiments, the anti-CD47 agent of the disclosure is administered to a human patient at a dose of 30 mg/kg weekly for at least nine weeks. In some embodiments, the anti-CD47 agent of the disclosure is administered to a human patient at a dose of 45 mg/kg weekly for at least nine weeks.

In some embodiments a primer agent is administered prior to administering a therapeutically effective dose of an anti-CD47 agent to the subject. In some embodiments, a primer agent is administered at a sub-therapeutic dose of an anti-CD47 agent of the disclosure. In some embodiments, a sub-therapeutic dose may be, for example, less than about 10 mg/kg, less than about 7.5 mg/kg, less than about 5 mg/kg, less than about 2.5 mg/kg, and may be less than or about 1 mg/kg. In some embodiments, a primer agent is an erythropoiesis-stimulating agent (ESA). In some embodiments, a primer agent is a priming dose of an anti-CD47 agent. Following administration of the priming agent, and allowing a period of time effective for an increase in reticulocyte production, a therapeutic dose of an anti-CD47 agent is administered. Administration may be made in accordance with the methods described in U.S. Pat. No. 9,623,079, herein specifically incorporated by reference.

In some embodiments, the anti-CD47 agent of the disclosure of pharmaceutical composition of the disclosure is administered, intravenously, orally, or subcutaneously. In some embodiments, the anti-CD47 agent of the disclosure of pharmaceutical composition of the disclosure is administered intravenously. In some embodiments, the anti-CD47 agent of the disclosure of pharmaceutical composition of the disclosure is administered orally. In some embodiments, the anti-CD47 agent of the disclosure of pharmaceutical composition of the disclosure is administered subcutaneously. In some embodiments, the anti-CD47 agent of the disclosure of pharmaceutical composition of the disclosure is administered by intravenous injection or infusion. Methods are provided for treating or reducing vascular inflammation by administering an anti-CD47 agent, alone or in combination with a statin, to a human in a dose that decreases vascular inflammation. In some embodiments the individual is monitored for indicia of vascular inflammation. In some embodiments, the individual is not genotyped for a 9p21 risk allele.

Effective doses of the therapeutic entity of the present invention vary depending upon many different factors, including the nature of the agent, means of administration, target site, physiological state of the patient, whether the patient is human or an animal, other medications administered, and whether treatment is prophylactic or therapeutic. Usually, the patient is a human. Treatment dosages can be titrated to optimize safety and efficacy.

In some embodiments, an effective dose of anti-CD47 agent and statin decreases the plaque area as a measure of total vessel area. For instance, the plaque area may be reduced by at least 2.5%, at least 5%, at least 7.5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or up to about 90%, compared to the absence of invention.

In some embodiments, an effective dose of anti-CD47 agent and statin decreases the necrotic core as a measure of the % of intima area by at least 2.5%, at least 5%, at least 7.5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or up to about 90%, compared to the absence of invention.

In some embodiments, an effective dose of anti-CD47 agent and statin decreases the necrotic core as a measure of the % of intima area. For instance, the necrotic core may be reduced by at least 2.5%, at least 5%, at least 7.5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or up to about 90%, compared to the absence of invention.

In some embodiments, an effective dose of anti-CD47 agent and statin increases the rate of efferocytosis. For instance, the efferocytosis rate may be increased by at least 2.5%, at least 5%, at least 7.5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or up to about 90%, compared to the absence of invention.

In some embodiments, the therapeutic dosage of a statin or an anti-CD47 agent can range from about 0.0001 to 500 mg/kg, and more usually 1 to 50 mg/kg, of the host body weight. The dosage may be adjusted for the molecular weight of the reagent.

An exemplary treatment regime entails administration daily, semi-weekly, weekly, once every two weeks, once a month, etc. For instance, the treatment regime may comprise administering an anti-CD47 agent and a statin once per day, once every other date, once every 2 days, once every 3 days, once every 4 days, once every 5 days, once every 6 days, weekly, once every two weeks, once a month, etc. In some embodiments, the treatment regime may comprise administering an anti-CD47 agent and statin individually at a separate treatment regime. For instance, an anti-CD47 agent may be administered once per day, once every other date, once every 2 days, once every 3 days, once every 4 days, once every 5 days, once every 6 days, weekly, once every two weeks, once a month, etc., whereas a statin may be administered once per day, once every other date, once every 2 days, once every 3 days, once every 4 days, once every 5 days, once every 6 days, weekly, once every two weeks, once a month, etc., wherein the statin is administered in a therapeutic regime that is different from the anti-CD47 agent. In another example, treatment can be given as a continuous infusion. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient. It will be understood by one of skill in the art that such guidelines will be adjusted for the molecular weight of the active agent, e.g. in the use of polypeptide fragments, in the use of antibody conjugates, in the use of high affinity SIRPα reagents, etc. The dosage may also be varied for localized administration, e.g. intranasal, inhalation, etc., or for systemic administration, e.g. i.m., i.p., iv., and the like.

For the treatment of disease, the appropriate dosage of the agent will depend on the severity and course of the disease, whether the agent is administered for preventive purposes, previous therapy, the patient's clinical history and response to the antibody, and the discretion of the attending physician. The agent is suitably administered to the patient at one time or over a series of treatments.

Therapeutic formulations comprising one or more agents of the invention are prepared for storage by mixing the agent having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the agent to be administered will be governed by such considerations, and is the minimum amount necessary to treat or prevent atherosclerosis.

The agent can be administered by any suitable means, including topical, oral, parenteral, subcutaneous, intraperitoneal, intrapulmonary, and intranasal. Parenteral infusions include intramuscular, intravenous, intraarterial, intraperitoneal, intrathecal or subcutaneous administration. In addition, the agent can be suitably administered by pulse infusion, particularly with declining doses of the agent.

The agent need not be, but is optionally formulated with one or more agents that potentiate activity, or that otherwise increase the therapeutic effect. These are generally used in the same dosages and with administration routes as used hereinbefore or about from 1 to 99% of the heretofore employed dosages.

An agent is often administered as a pharmaceutical composition comprising an active therapeutic agent and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear an anti-CD47 agent by non-covalent associations, such as non-covalent bonding or by encapsulation. The nature of the carrier can be either soluble or insoluble for purposes of the invention. Those skilled in the art will know of other suitable carriers for binding anti-CD47 agents, or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Carriers and linkers specific for radionuclide agents include radiohalogenated small molecules and chelating compounds. A radionuclide chelate may be formed from chelating compounds that include those containing nitrogen and sulfur atoms as the donor atoms for binding the metal, or metal oxide, radionuclide.

Radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the methods of the invention. Such moieties may be conjugated to the anti-CD47 agent through an acceptable chemical linker or chelation carrier. Positron emitting moieties for use in the present invention include ¹⁸F, which can be easily conjugated by a fluorination reaction with the agent.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Vascular ¹⁸F-FDG Uptake after Treatment with the Macrophage Checkpoint Inhibitor Magrolimab

Macrophage checkpoint inhibition represents a new paradigm in immuno-oncology. This approach reactivates the phagocytic clearance of cancer cells to prevent tumor growth. However, the defective clearance of inflamed tissue is also now recognized as a hallmark of atherosclerotic cardiovascular disease, and a potential therapeutic target. Here we report analyses of ¹⁸F-FDG-PET/CT scans from mice and humans treated with the first macrophage checkpoint inhibitor, magrolimab. Subjects receiving this drug demonstrated a reduction in arterial ¹⁸F-FDG uptake, which reflects an improvement in vascular inflammation.

Atherosclerosis is the process underlying heart attack and stroke, and is the leading cause of death worldwide. It is characterized by the accumulation of diseased and dying macrophages and smooth muscle cells in the vessel wall. Normally, pathological cells such as these would be identified for phagocytic removal by macrophages in the plaque (a process known as “efferocytosis”). However, this process is defective in atherosclerosis, due in part to the pathological upregulation of a so-called ‘don't eat me’ molecule known as CD47.

Intriguingly, the same anti-phagocytic markers found in atherosclerosis have now been shown to be overexpressed by a wide variety of cancers. These signals allow malignant cells to evade macrophage clearance and permit tumor growth. Translational efforts in the field of immuno-oncology have thus focused on targeting these dominant macrophage checkpoint regulators, with the goal of reactivating immune surveillance and accelerating tumor clearance. Recently, the first human trial of a humanized anti-CD47 antibody (termed magrolimab) was conducted in patients with aggressive and indolent lymphoma who had become refractory to rituximab alone or in combination with chemotherapy. In this study, the macrophage checkpoint inhibitor magrolimab showed promising results, quantified by reductions in tumor burden measured by ¹⁸F-FDG-PET/CT.

Of note, the ¹⁸F-FDG-PET signal used to detect the high metabolic activity of cancer cells is not specific to primary tumors and metastases. Indeed, this imaging modality also correlates with burden of atherosclerosis, and has been used to quantify vascular inflammation and response to therapy in humans. The impact of CD47 inhibition on vascular disease was assayed in a murine model of atherosclerosis. Motivated by these results, we then performed a retrospective analysis of the ¹⁸F-FDG-PET/CT scans performed at our institution as part of the first human studies of magrolimab. Our goal was to determine if blockade of CD47 could reduce vascular inflammation in these individuals, and ascertain whether additional prospective studies may be justified.

### Methods

Animals and diet. Male ApoE^{tm1Unc} (ApoE^{-/-}) mice on a C56BL/6 background were purchased from the Jackson Laboratory (Bar Harbor, ME). During the experimental period, all animals were fed a high-fat diet (21% anhydrous milk fat, 19% casein, and 0.15% cholesterol, Dyets Inc., Bethlehem, PA). Animal studies were approved by the Stanford University Administrative Panel on Laboratory Animal Care (protocol #27279) and conformed to the NIH guidelines for the care and use of laboratory animals.

Animal model and in vivo interventions. Eight week old mice were fed a high-fat diet for two weeks. Then, a shear stress modifier (referred to as a cast) was surgically placed over the right carotid artery to induce a vulnerable lesion, described previously. For the ensuing 9 weeks, mice were fed a high-fat diet and randomly assigned to receive 200 μg of the inhibitory anti-CD47 antibody (MIAP410, Lot #705318N1, BioXCell, Lebanon, NH) IP QOD or IgG1 control (MOPC-21, LOT #61991601B, BioXCell, Lebanon, NH). PET/CT imaging was conducted 6 weeks (for aortic quantification) and 9 weeks (for carotid quantification) after surgery.

Murine ¹⁸F-FDG-PET/CT scan and analysis. Mice were fasted overnight prior to each scan. Mice were anesthetized with isoflurane, and special precautions were taken to maintain body temperature. The radiotracer (15-20 MBq of ¹⁸F-FDG; Stanford Cyclotron & Radiochemistry Facility) was administered intravenously to the mice. In addition, a long circulating formulation of iodinated triglyceride (Fenestra VC, MediLumine, Montreal, Quebec) or colloidal gold (Mvivo Au, particle size 15 nm, MediLumine, Montreal, Quebec) was used as a contrast agent. 3 h after ¹⁸F-FDG administration, the mice were placed on the bed of a dedicated small animal PET/CT scanner (Inveon PET/CT, Siemens Medical Solution, Malvern, PA), and a static PET scan (30 minutes) was obtained. All images were reconstructed using 3D-OSEM. The same acquisition bed was used for the CT scan. The CT system was calibrated to acquire 720 projections (voltage 80 kV; current 500 μA), with a voxel size of 0.103×0.103×0.103 mm³. Quantitative analysis was performed using Inveon Research Workplace 4.2 software (Ed4.2.0.15, Siemens, Malvern, PA). ¹⁸F-FDG uptake was quantified in a 3 mm³ volume of interest upstream (caudally) from the cast on the carotid artery. ¹⁸F-FDG uptake in the thoracic aorta was quantified by drawing a 3D region of interest on the axial slices from the CT scan followed by ROI interpolation. The standardized uptake values (SUV) were calculated and the mean value was used.

Tissue preparation and histological analysis. Mice were perfused with PBS via cardiac puncture and then perfusion fixed with 4% phosphate-buffered paraformaldehyde. The entire aortic arch with the origins of the right and left common carotid artery were carefully collected, embedded in optimal cutting temperature compound (Catalog #25608-930, VWR), and sectioned using a cryostat (Leica CM 1950, Buffalo Grove, IL). In the carotid artery sections, plaque volume (in mm³) was quantified by hematoxylin and eosin staining (Catalog #SH26-500D and SE22-500D, Thermo Fisher Scientific). Histological sections were imaged using a Zeiss Axioplan (equipped with a Nikon camera). Sections were analyzed using Image J/FIJI software (Version: 2.0.0/1.52p, NIH) in a blinded fashion.

Study population and design. The 13 participants enrolled in the first-in-human clinical trials of magrolimab at Stanford University were identified for inclusion in this retrospective analysis. These patients had refractory or relapsed B-cell lymphoma and had become refractory to rituximab alone or in combination with chemotherapy prior to enrollment. The protocol was reviewed and approved by the institutional review board at Stanford University (IRB #55497). Participants were treated with magrolimab in combination with background rituximab therapy. Rituximab was administered intravenously at a dose of 375 mg per square meter of body surface area, weekly in cycle 1 starting in week 2, and then monthly in cycles 2 through 6². Magrolimab was administered intravenously with a priming dose of 1 mg per kilogram of body weight, followed by weekly doses of 20 to 45 mg per kilogram. Baseline and follow-up ¹⁸F-FDG-PET/CT scans were available for all study patients and were reviewed by 2 Nuclear Medicine physicians blinded to other examinations but aware of the protocol. Four scans were deemed uninterpretable for vascular uptake due to extensive cervical lymphadenopathy and these patients were excluded.

¹⁸F-FDG-PET/CT scans and analysis. All patients underwent ¹⁸F-FDG-PET/CT before and at regular intervals after the administration of magrolimab. Baseline ¹⁸F-FDG-PET/CT scans were obtained 12 days (mean±SD: 12.1±9.8) before therapy initiation. The first follow-up PET scans were performed 63 days (mean±SD: 62.6±33.5) after therapy initiation. Patients fasted for a minimum of 6 hours before intravenous ¹⁸F-FDG administration. The time from injection to the start of the PET/CT scans was 72 minutes (mean±SD: 71.7±19.6). The baseline and first restaging PET/CT images were obtained in 3D mode from the vertex to the toes. The activity of ¹⁸F-FDG administered ranged from 7.9 to 11.3 mCi (mean±SD: 9.8±1.1). PET/CT scans were acquired following procedure standards on Discovery 690, 710, or MI scanners (GE Healthcare, Waukesha, WI) in use at our institution. Both pre- and post-treatment scans were done using the same scanner. Images were anonymized and analyzed using MIM Vista version 6.9.2 (MIM Software Inc., Cleveland, OH). The analyses were performed as previously described, and vascular uptake was quantified in the carotid arteries to avoid confounding by signal present in the mediastinal lymph nodes. Briefly, the carotid artery bifurcations on both sides were identified and arterial FDG uptake was measured starting 2 cm below the carotid artery bifurcation and continuing superiorly to 2 cm into the internal carotid artery. Measurements were made in the axial plane and maximum standardized uptake values (SUV) were obtained. The maximum target-to-background ratio (TBR) was calculated (ratio of the maximum SUV of the artery compared to background activity in the ipsilateral internal jugular vein). Next, the carotid artery with the highest FDG uptake was identified as the index vessel. The most diseased segment represented the arterial segment with the highest ¹⁸F-FDG uptake at baseline scan. This was calculated as an average maximum TBR derived from four contiguous axial segments. Additionally, CT data was used to analyze the coronary calcium score using Horos software (Horos Project).

Statistical analysis. Statistical analyses were performed using GraphPad Prism 8 (GraphPad Inc., San Diego, CA). Data are presented as mean±standard deviation (SD). Data were tested for normality using D'Agostino-Pearson test and were analyzed using t-test and Mann-Whitney test (two-tailed). A p-value of 0.05 or less was considered to denote significance.

### Results

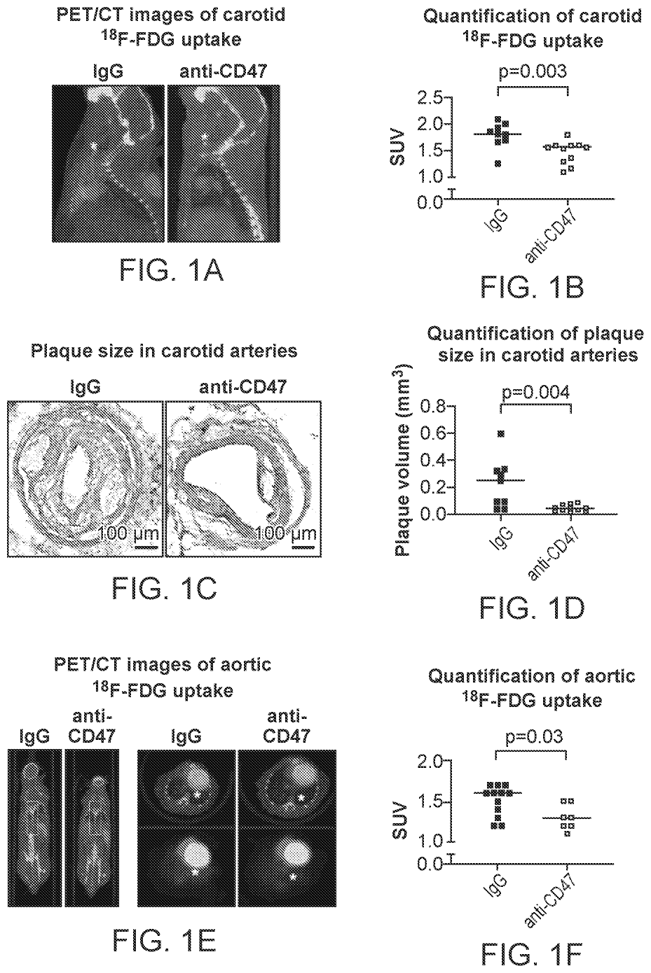

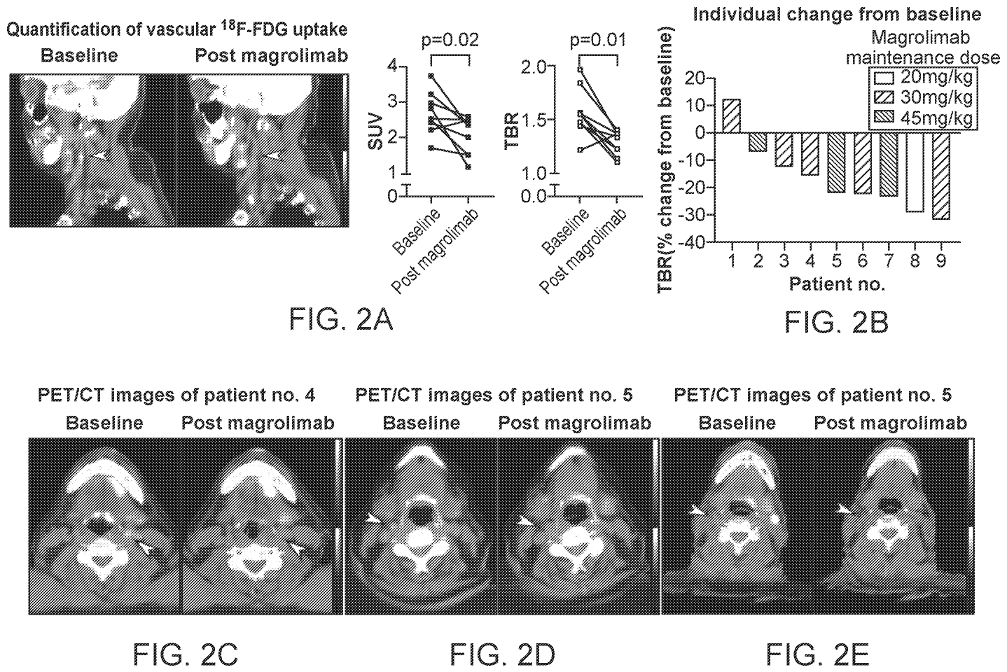

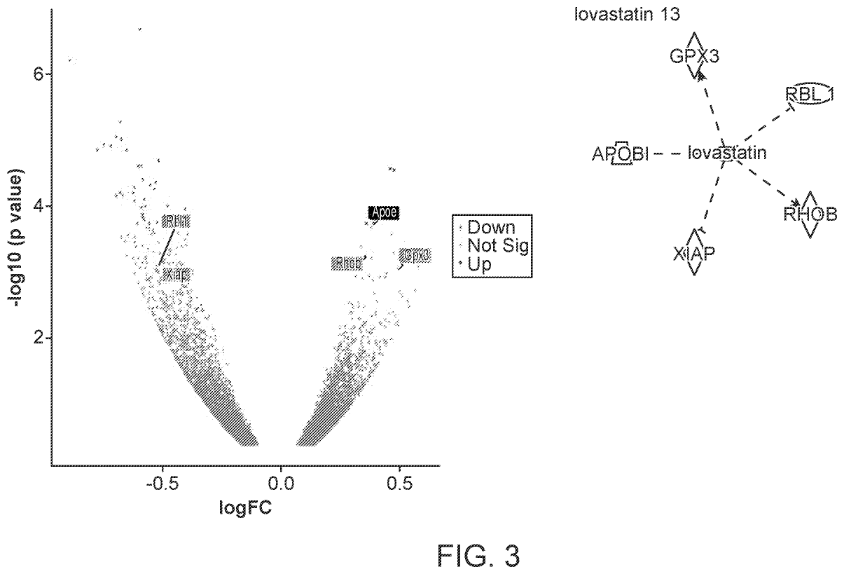

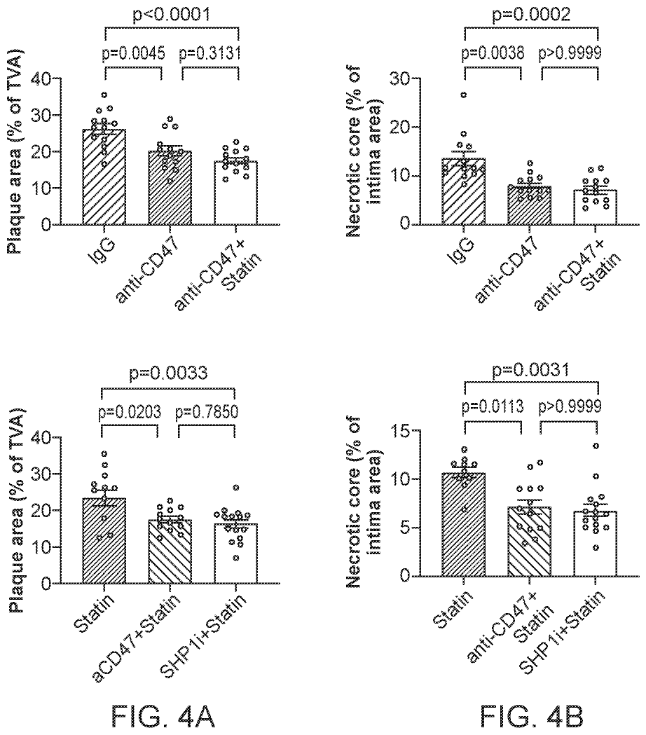

Drug effect on vascular ¹⁸F-FDG uptake in mice. ¹⁸F-FDG uptake was first quantified in advanced lesions from a carotid plaque vulnerability model. Here we observed a reduced ¹⁸F-FDG uptake measured by mean SUV (mean±SD: 1.79±0.24 versus 1.47±0.21; p=0.005 by unpaired t-test; FIG. 1A-B) in anti-CD47 treated mice compared to their respective controls. These mice demonstrated a reduction in carotid lesion burden by histopathology (FIG. 1C-D). To confirm these findings, we also assessed aortic ¹⁸F-FDG uptake and noted a significant improvement in this vascular bed as soon as six weeks after initiating anti-CD47 therapy (mean±SD: 1.51±0.19 versus 1.30±0.15; p=0.03 by Mann-Whitney test; FIG. 1E-F).

Baseline characteristics of patients. The baseline characteristics of the patients are shown in Table 1. The age ranged from 59 to 81 years (mean±SD: 71.0±7.3) and 22% of the patients were women. Of note, cardiovascular risk factors were common: 44% of the patients had diabetes mellitus and 89% had hypertension. Two thirds of the patients had atherosclerotic disease at baseline, and 22% had previously sustained a myocardial infarction. Approximately 44% of patients had been treated with statin. Overall, 78% (7 of 9) of the patients had coronary calcification present and 56% (5 patients) had a moderate to high risk coronary artery calcification score (mean±SD: 324±566 Agatston units).

Drug effect on vascular ¹⁸F-FDG uptake in humans. We observed a reduction in ¹⁸F-FDG uptake measured by maximum SUV (mean±SD: 2.75±0.58 versus 2.09±0.53; p=0.01 by paired t-test; FIG. 1A) and maximum TBR (mean±SD: 1.56±0.22 versus 1.28±0.11; p=0.006 by paired t-test; FIG. 1A) in the most diseased segment of the index vessel after magrolimab treatment.

This study evaluated the effect of macrophage checkpoint inhibitors on vascular inflammation. We observed that blockade of CD47 led to a reduction in arterial FDG uptake in mouse models of atherosclerosis as well as humans enrolled in a clinical trial. These improvements were noted as soon as nine weeks after treatment initiation. Together, these data provide the first human evidence that pro-efferocytic therapies favorably impact atherosclerotic cardiovascular disease.

Current pharmacological interventions for coronary artery disease mainly address traditional risk factors (e.g., hypertension and hyperlipidemia). To identify new translational targets, investigators have recently turned their attention to the “inflammatory hypothesis” of atherosclerosis, which has been bolstered by promising results with agents such as canakinumab (which targets the interleukin-1P immunity pathway). Efferocytosis signaling is also thought to occur independently of traditional risk factors, and has been directly linked to inflammation related to cytokines such as tumor necrosis factor-α. When coupled with prior pre-clinical studies, the human data provided herein demonstrate that reactivating macrophage phagocytosis can clear inflamed and apoptotic tissue from the plaque, and could reduce lesion vulnerability.

In conclusion, this is the first human evidence that the pro-efferocytic antibody, magrolimab, reduces arterial ¹⁸F-FDG uptake. These results provide a rationale for prospective, randomized, placebo-controlled cardiovascular trials. Macrophage checkpoint inhibition provides a new orthogonal therapy for individuals suffering from atherosclerotic vascular disease.

TABLE 1
Baseline characteristics of the 9 patients
included in the retrospective analysis.

| Characteristic | All patients (n = 9) | |
|---|---|---|
| Mean age (SD) - yr | 71.0 | (7.3) |
| Sex - no. (%) | | |
| Male | 7 | (77.8) |
| Female | 2 | (22.2) |
| Mean body-mass index (SD)* | 30.2 | (7.1) |
| Race - no. (%)† | | |
| White | 7 | (77.8) |
| Other | 2 | (22.2) |
| Risk factors and coexisting conditions - no. (%) | | |
| Hypertension | 8 | (88.9) |
| Hyperlipidemia | 5 | (55.6) |
| Diabetes mellitus | 4 | (44.4) |
| Insulin therapy | 3 | (33.3) |
| Current smoker | 0 | (0) |
| Atherosclerotic disease‡ | 6 | (66.7) |
| Prior myocardial infarction | 2 | (22.2) |
| Medications - no. (%) | | |
| Statin | 4 | (44.4) |
| Previous rituximab therapy (alone | 9 | (100) |
| or in combination) - no. (%) | | |
| Mean time from magrolimab initiation | 62.6 | (33.5) |
| to PET/CT scan (SD) - days | | |
| Mean coronary artery calcification | 324 | (566) |
| score (SD) - Agatston units | | |
| Low risk - no. (%) | 4 | (44.4) |
| Moderate risk - no. (%) | 2 | (22.2) |
| High risk - no. (%) | 3 | (33.3) |

*The body-mass index is the weight in kilograms divided by the square of the height in meters.
†Race was reported by the patient.
‡Atherosclerotic disease includes coronary artery disease, carotid artery disease, and atherosclerotic aortic disease.

### REFERENCES

1. Kojima Y, Volkmer J P, McKenna K, et al. CD47-blocking antibodies restore phagocytosis and prevent atherosclerosis. Nature 2016; 536:86-90.
2. Advani R, Flinn I, Popplewell L, et al. CD47 Blockade by Hu5F9-G4 and Rituximab in Non-Hodgkin's Lymphoma. N Engl J Med 2018; 379:1711-21.
3. Tawakol A, Fayad Z A, Mogg R, et al. Intensification of statin therapy results in a rapid reduction in atherosclerotic inflammation: results of a multicenter fluorodeoxyglucose-positron emission tomography/computed tomography feasibility study. J Am Coll Cardiol 2013; 62:909-17.
4. Cheng C, Tempel D, van Haperen R, et al. Atherosclerotic lesion size and vulnerability are determined by patterns of fluid shear stress. Circulation 2006; 113:2744-53.
5. Libby P, Ridker P M, Maseri A. Inflammation and atherosclerosis. Circulation 2002; 105:1135-43.
6. Ridker P M, Everett B M, Thuren T, et al. Antiinflammatory Therapy with Canakinumab for Atherosclerotic Disease. N Engl J Med 2017; 377:1119-31.

### Example 2

### Synergy with Statins

RNA sequencing analysis revealed lovastatin as one of the top upstream regulators of the CD47/SIRP-alpha axis in macrophages in vitro. RNA sequencing was performed on bone marrow-derived mouse macrophages treated with a nanoparticle loaded with a chemical inhibitor of the Src homology 2 domain-containing phosphatase-1 (SHP-1) and thus interrupting the CD47/SIRP-alpha signaling axis. Using Ingenuity Pathway Analysis (Qiagen), lovastatin was one of the top upstream regulators, suggesting overlapping mechanism of action between the interruption of the CD47/SIRPalpha axis and statin signaling and thus additive effects on macrophages in preventing atherosclerosis.

The combination of pro-efferocytic therapies (anti-CD47 antibodies or nanoparticles loaded with a SHP1-inibitor) and atorvastatin treatment showed additive or synergistic effects on atherosclerotic plaque burden in vivo. Atheroprone apolipoprotein-E-deficient mice were treated with (1) IgG isotype control antibodies, (2) anti-CD47 antibodies, (3) atorvastatin, (4) the combination of anti-CD47 antibodies and atorvastatin, and (5) the combination of the nanoparticle loaded with a SHP1-inhibitor and atorvastatin. Additive or synergistic effects on plaque burden were measured as plaque area in % of total vessel area in mice treated with the combination of pro-efferocytic therapies and atorvastatin. Additionally, the necrotic core size (measured as necrotic core in % of intima area) was significantly reduced in the cohorts treated with a combined regimen.

### Example 3

### Statins Amplify the Anti-Atherosclerotic Effects of Pro-Phagocytic Therapies

### RNA Sequencing Identified HMG-CoA Reductase Inhibitor as One of the Top Upstream Regulators of SHP-1 Inhibition in Macrophages.

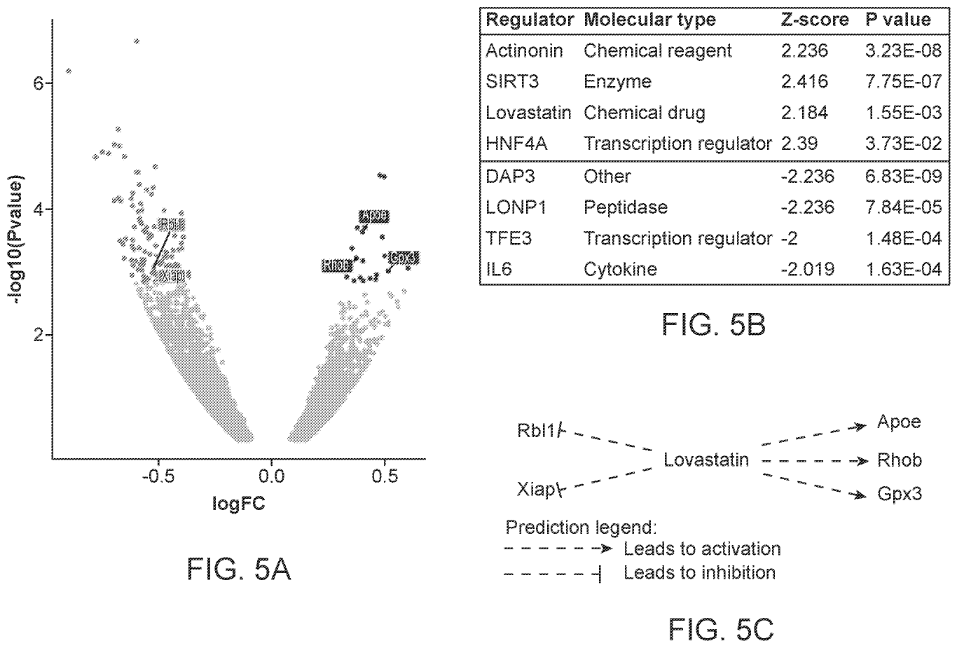

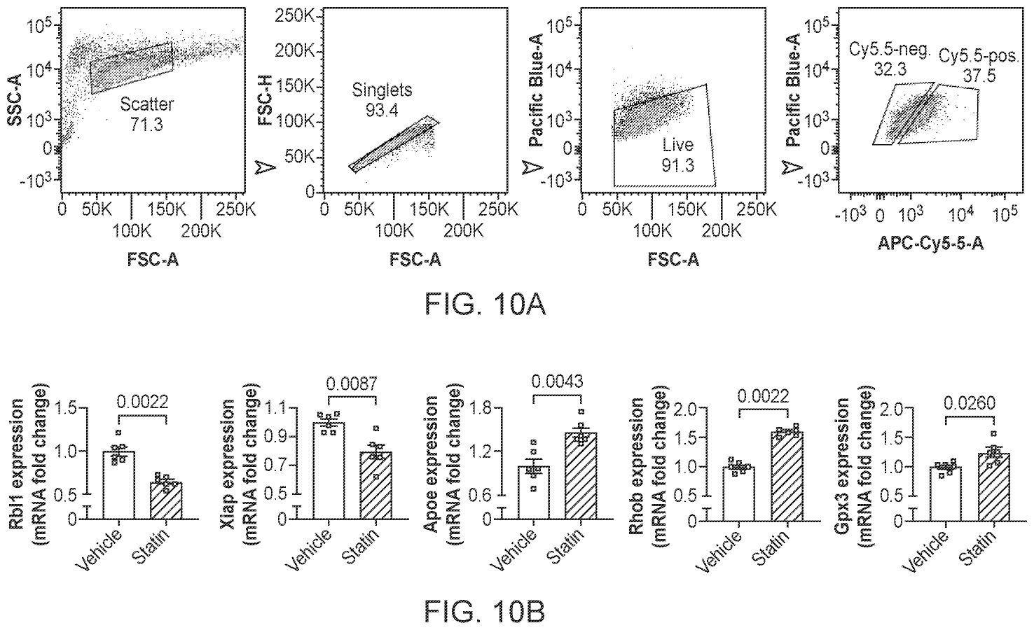

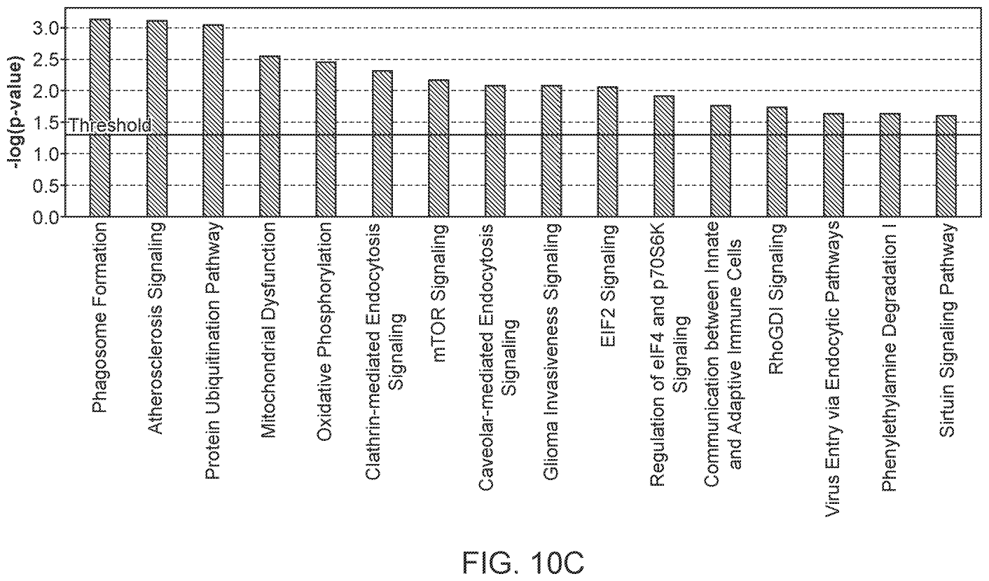

RNA sequencing was used to examine the transcriptome of macrophages after CD47-SIRPα axis blockade. Bone marrow-derived macrophages were incubated with SWNT or SHP1i for 24 hours, and sorted by flow cytometry to isolate Cy5.5-positive macrophages in each group, which were then subjected to RNA sequencing (FIG. 10a). 128 differentially expressed genes were identified with a false-discovery rate of less than 0.10 (19 up regulated and 109 down-regulated) in this study (FIG. 5a).

“Upstream regulators” were identified using Qiagen's Ingenuity Pathway Analysis. Lovastatin, a first-generation HMG-CoA reductase inhibitor, was one of the top activated upstream regulators and the only drug in the database (z15 score 2.184), based on the relevant regulation of apolipoprotein E, ras homolog family member B, RB transcriptional corepressor like 1, glutathione peroxidase 3, and X-linked inhibitor of apoptosis (FIG. 5b-5c and Table 2). These findings were validated for atorvastatin, the most widely prescribed statin with one of the most favorable safety profiles, by quantitative polymerase chain reaction. Similar gene expression changes were found upon atorvastatin treatment (FIG. 10b). In conclusion, these data suggested an unexpected overlap of HMG-CoA reductase inhibition and CD47-SIRPα blockade.

TABLE 2
Upstream regulators predicted by Ingenuity Pathway Analysis.
Filter criteria: significant Z-score (³2 for predicted
activation and 1 £ -2 for predicted inhibition). Sorting
criteria: P value of overlap.

| Upstream | Molecular | Predicted | | P-value |
|---|---|---|---|---|
| regulator | type | state | Z-score | overlap |
| Actinonin | Chemical | Activated | 2.236 | 3.23E−08 |
| | reagent | | | |
| SIRT3 | Enzyme | Activated | 2.416 | 7.75E−07 |
| Lovastatin | Chemical drug | Activated | 2.184 | 1.55E−03 |
| HNF4A | Transcription | Activated | 2.39 | 3.73E−02 |
| | regulator | | | |
| DAP3 | Other | Inhibited | −2.236 | 6.83E−09 |
| LONP1 | Peptidase | Inhibited | −2.236 | 7.84E−05 |
| TFE3 | Transcription | Inhibited | −2 | 1.48E−04 |
| | regulator | | | |
| IL6 | Cytokine | Inhibited | −2.019 | 1.63E−04 |
| TLR4 | Transmembrane | Inhibited | −2.412 | 3.93E−04 |
| | receptor | | | |
| CD3 | Complex | Inhibited | −2.425 | 1.07E−03 |
| CD44 | Other | Inhibited | −2.352 | 1.27E−03 |
| SYVN1 | Transporter | Inhibited | −2.236 | 1.33E−03 |
| CD24 | Other | Inhibited | −2 | 3.40E−03 |
| LIF | Cytokine | Inhibited | −2.236 | 8.09E−03 |
| STAT3 | Transcription | Inhibited | −2.739 | 2.37E−02 |
| | regulator | | | |
| Pirinixic acidd | Chemical | Inhibited | −2.382 | 4.24E−02 |
| | toxicant | | | |
| Insulin | Group | Inhibited | −2.449 | 5.95E−02 |
| ESR1 | Ligand- | Inhibited | −2.465 | 1.04E−01 |
| | dependent | | | |
| | nuclear receptor | | | |

### Combined Treatment of CD47-SIRPα Blockade and Atorvastatin Showed Additive Effects on Atherosclerotic Plaque Activity In Vivo.

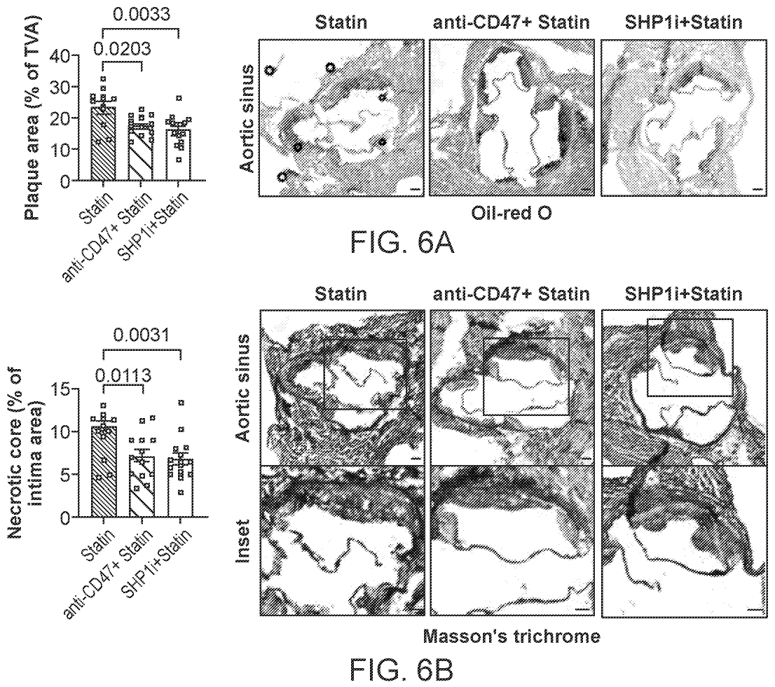

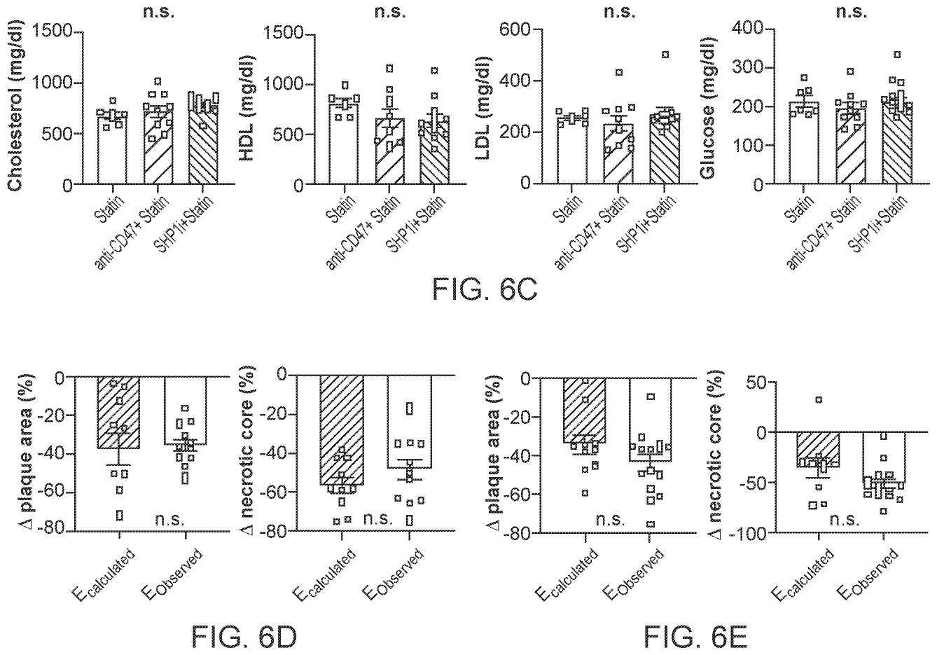

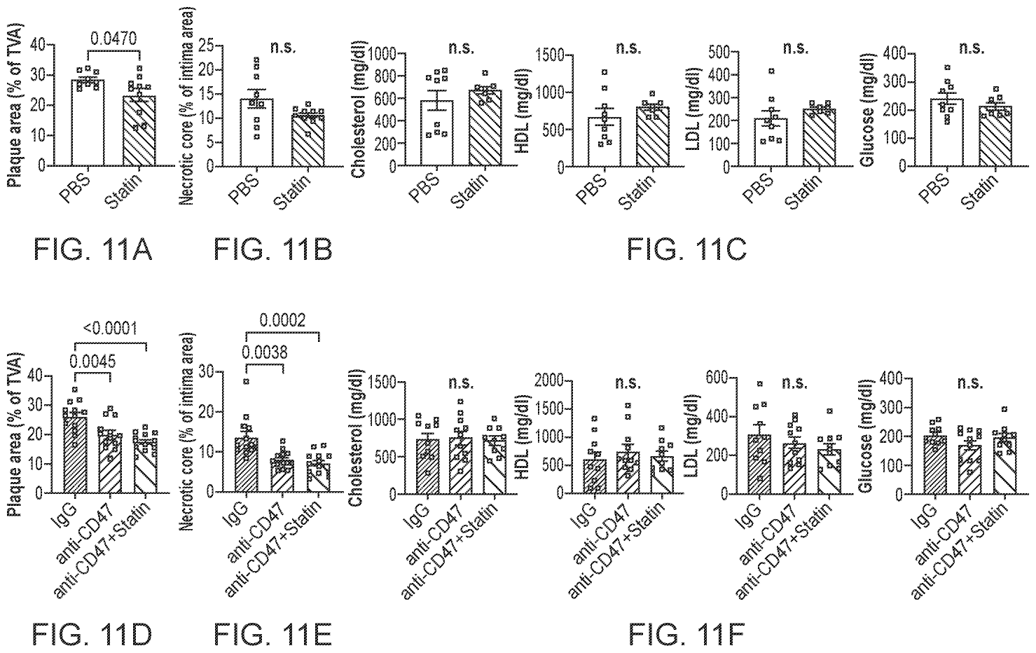

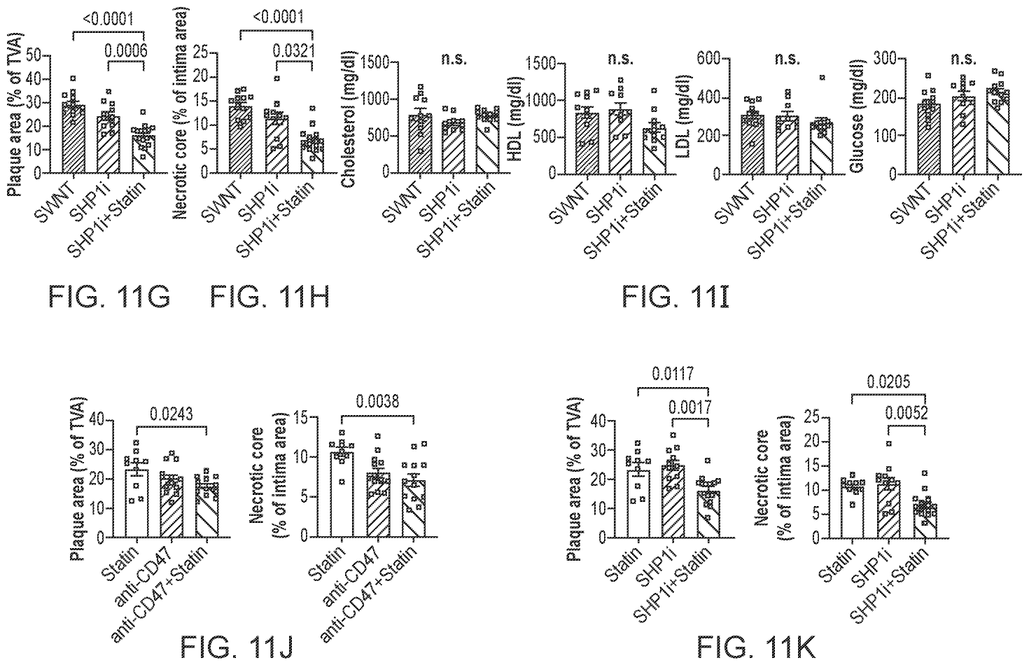

To test whether combined treatment of CD47-SIRPα blockade and HMG CoA reductase inhibition has additive effects on the atherosclerotic plaque activity in vivo, high-fat diet-fed Apoe−/− mice received therapy with atorvastatin alone or in combination with CD47-SIRPα blockade (FIG. 11a-11i). The latter was achieved by targeting either CD47 (using anti-CD47 antibodies) or SIRPα's downstream effector molecule SHP-1 (using SHP1i). Combined treatment not only decreased lesion size but also reduced necrotic core area (FIG. 6a-6b). Without being bound by theory, it is believed that the necrotic core is a key driver for plaque vulnerability in lesions and thus for acute vascular events. There were no significant differences in plasma cholesterol and blood glucose between the cohorts (FIG. 6c). Subsequently, the single treatment cohorts were used to determine additivity/synergy of compounds (FIG. 11j-11k). Applying the Bliss independence model on the analyses of lesion area and necrotic core size, an additive anti-atherosclerotic effect was computed for both parameters in vivo (FIG. 6d-6e). FIG. 6d provides the additivity for anti-CD47 and statin combination therapy. FIG. 6e provides the additivity for SHP1i and statin combination therapy.

Together, these observations provide evidence of an additive therapeutic effect upon combined treatment of CD47-SIRPα blockade and HMG CoA reductase inhibition.

### Combined Treatment of CD47-SIRPα Blockade and Atorvastatin Showed Additive Effects on Efferocytosis Rate In Vitro and In Vivo.

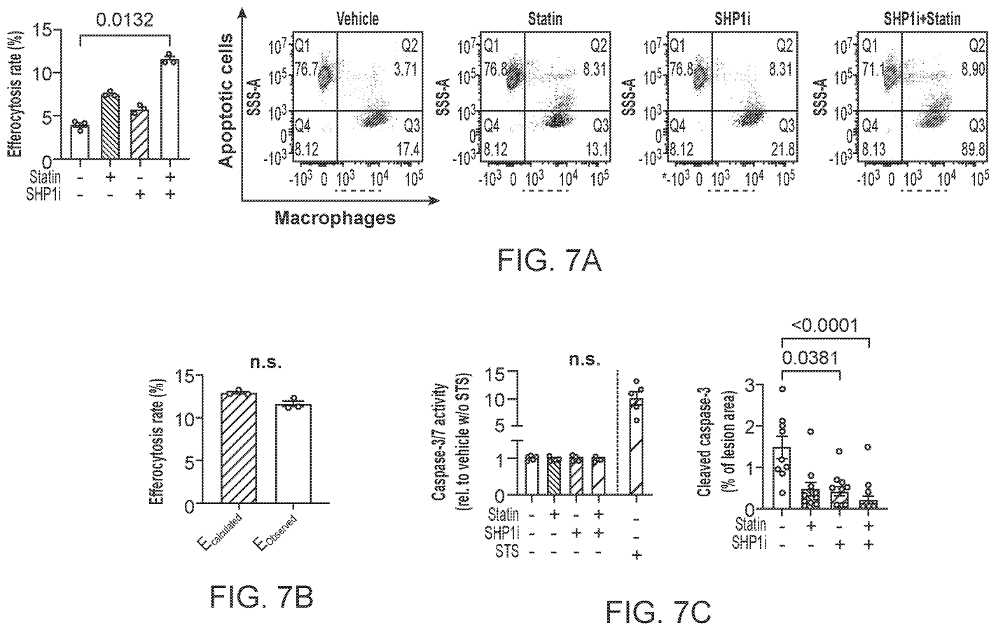

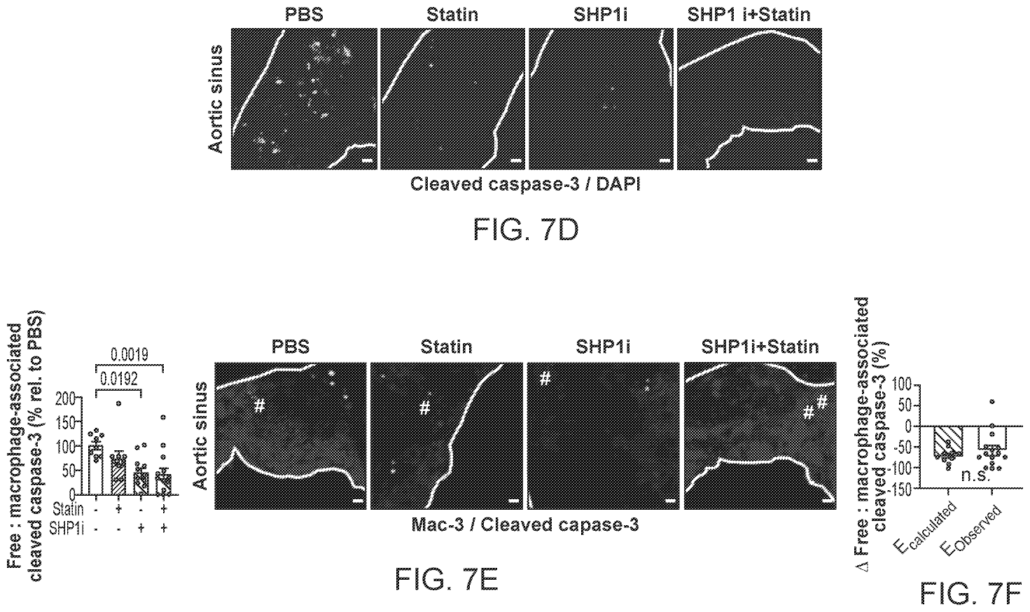

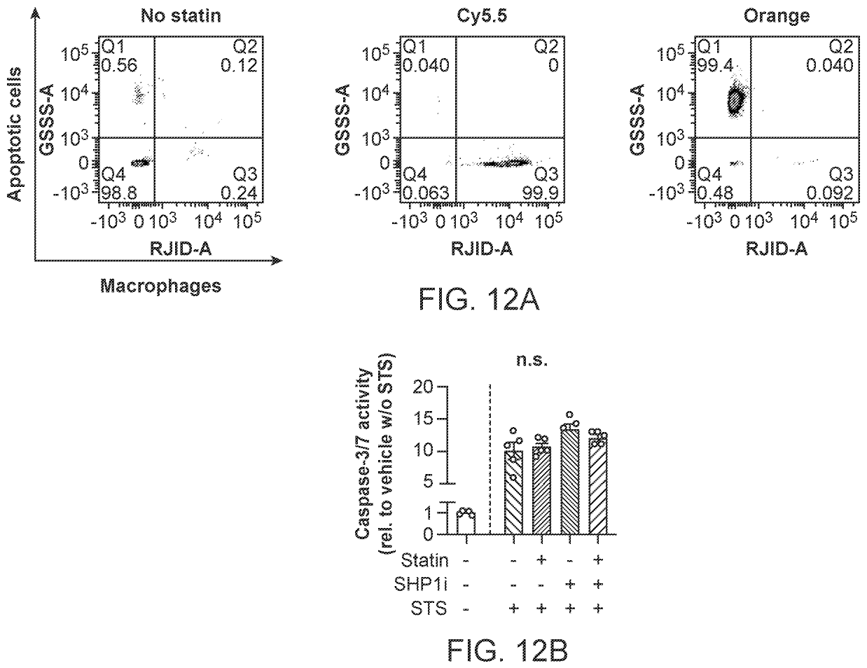

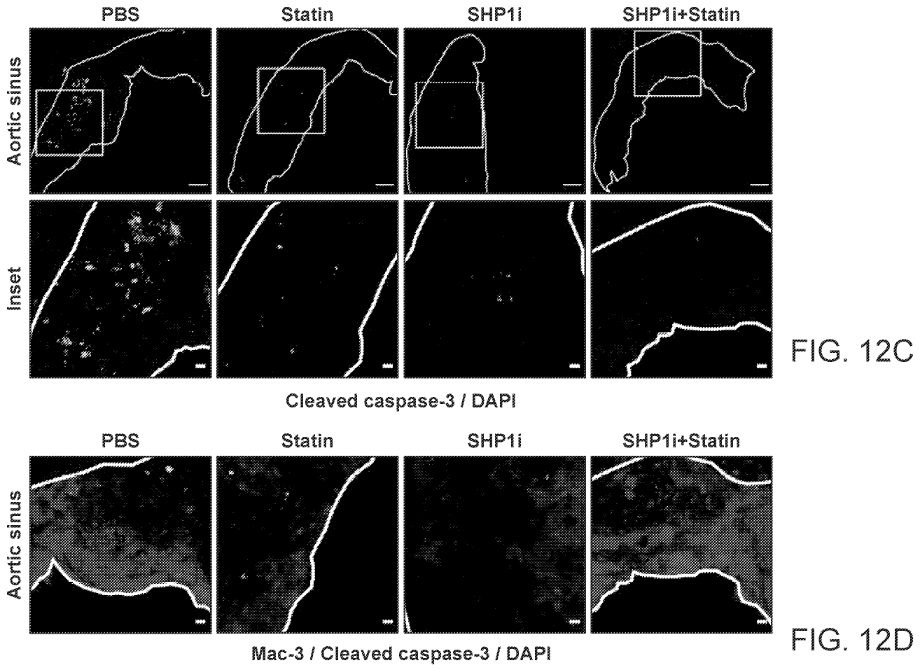

To test whether treatment with atorvastatin increases the efferocytosis rate and/or benefits lesion development in atherosclerosis, an in vitro phagocytosis assay was employed. Using flow cytometry, a relevant increase of the efferocytic rate of apoptotic cells was observed upon combined treatment (atorvastatin plus SHP1i) compared to single therapies (FIG. 7a and FIG. 12a). Bliss independence model confirmed additivity (FIG. 7b). Inhibition of the CD47-SIRPα axis (using anti-CD47 antibodies or SHP1i) did not alter the rate of programmed cell death in our cells. Similarly, an effect on apoptosis by atorvastatin or combined treatment strategies was not found (FIG. 7c and FIG. 12b), suggesting an enhancement of efferocytosis without altering apoptosis.

To determine the relevance of these observations in vivo, the cleaved caspase-3 activity was also investigated and the number of “free” apoptotic bodies not associated with an intraplaque macrophage, both reliable measures of accumulation of apoptotic bodies and thus efferocytosis in tissue specimens. In agreement with the in vitro observations, a decrease in the number of apoptotic bodies was found in the lesion with the combined treatment, as suggested by our immunofluorescence studies (FIG. 7d-7e and FIG. 12c-12d). Again, Bliss independence model demonstrated additivity (FIG. 7f). Taken together, these data suggested that the combination of HMG-CoA reductase inhibition and CD47-SIRPα blockade markedly increased the efferocytosis rate and thus may explain the additive effect on atherosclerotic plaque activity.

In sum, the foregoing data provide evidence that atrovastain is directly linked to the “don't eat me” molecule, CD47, and thus, to the removal of apoptotic debris, supporting a causal relationship.

### Atorvastatin Inhibited NFκB1 p50 Nuclear Translocation Under Atherogenic Conditions and Thus Directly Regulated Gene Expression of CD47.

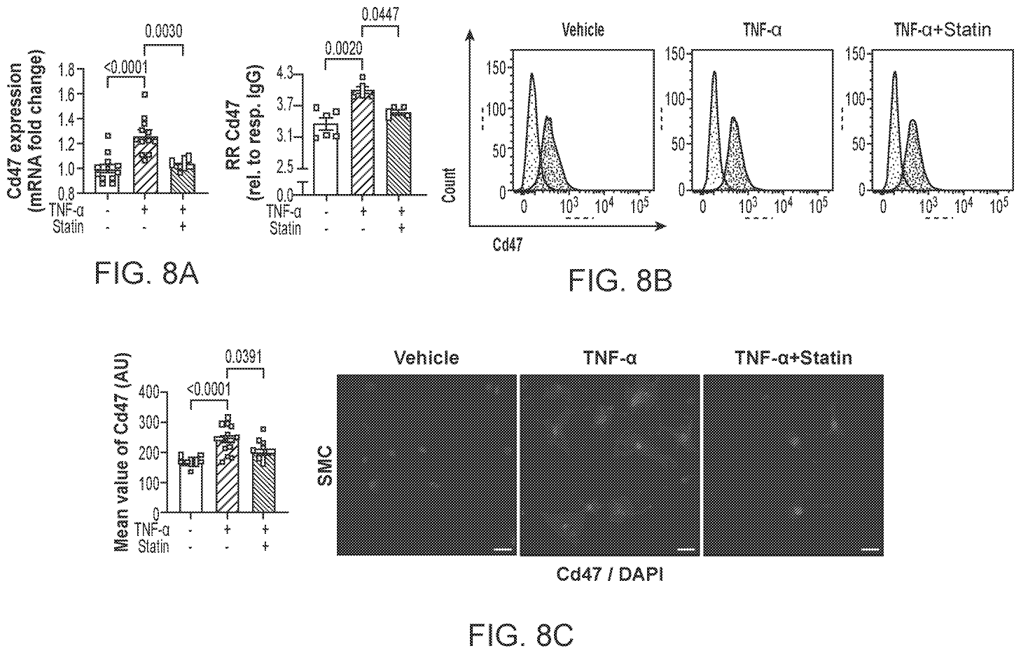

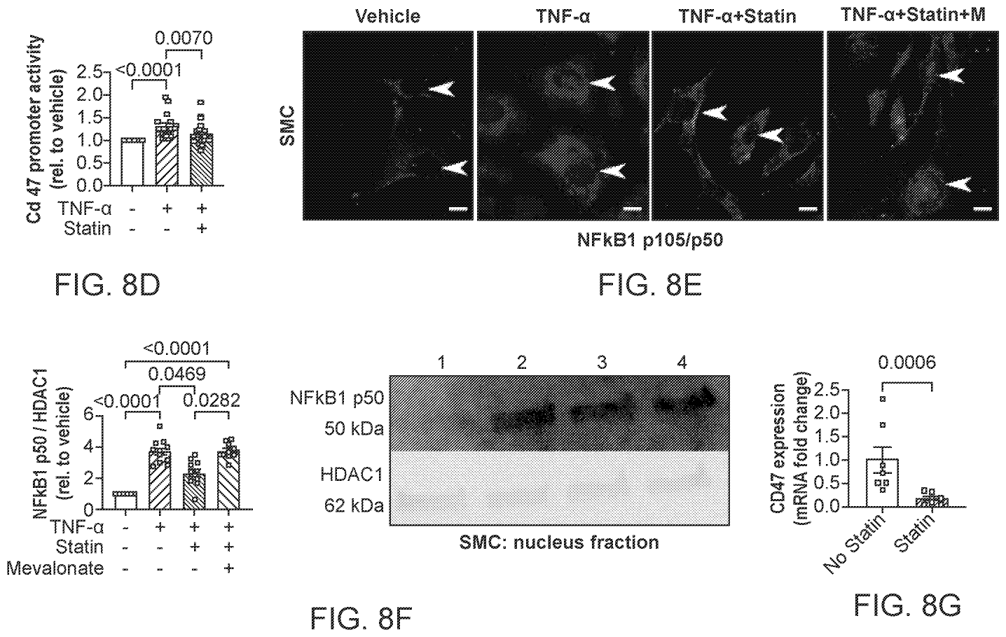

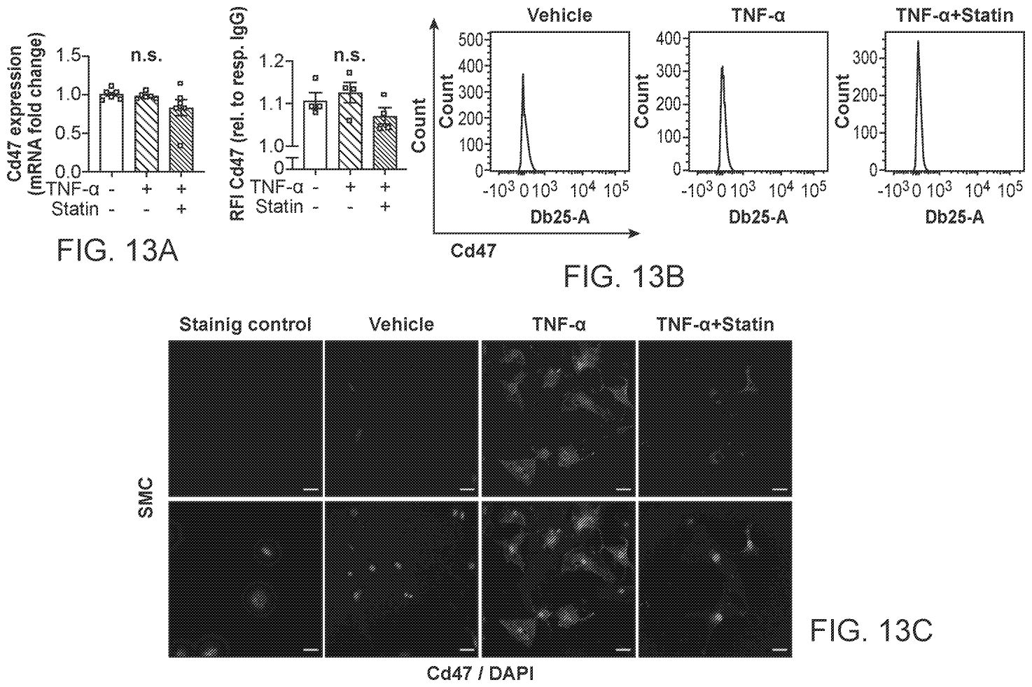

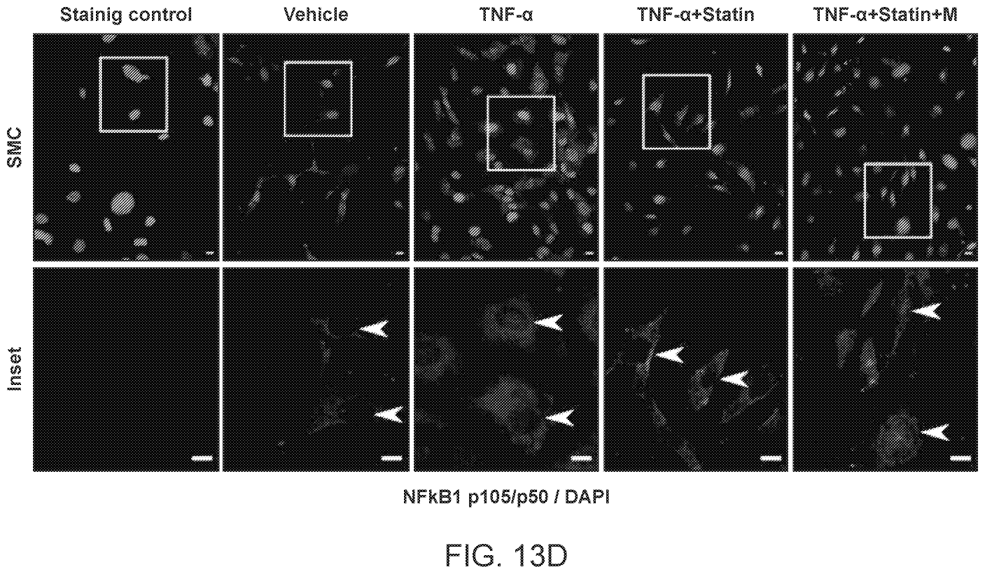

Having identified the efferocytic rate (discussed above) as a pivotal link for additivity of combined treatment, the underlying mechanism was then investigated. To test whether there is a direct effect of atorvastatin on the expression of the “don't-eat-me” molecule, CD47, in atherosclerosis and efferocytosis, CD47 expression was investigated in two of the major cellular components of atherosclerosis, smooth muscle cells and macrophages. Stimulation with tumor necrosis factor-α increased CD47 expression, but interestingly this effect was more pronounced in smooth muscle cells compared to macrophages. Treatment with atorvastatin resulted in a larger reduction of CD47 on both RNA and protein levels in smooth muscle cells (FIG. 8a-8c and FIG. 13a-13c).

To assay for a direct link between atorvastatin treatment and CD47 expression in smooth muscle cells, a luciferase reporter assay was used. It was observed that atorvastatin was able to inhibit the tumor necrosis factor-α induced CD47 promoter activity (FIG. 8d). It was found that atorvastatin inhibited the nuclear translocation of NFκB1 p50, a key transcriptional factor for CD47. Importantly, the effect was eliminated with the addition of mevalonate, an antagonist to atorvastatin (FIG. 8e-8f and FIG. 13d). These data demonstrated that atorvastatin directly reduced the pathological CD47 upregulation in atherosclerosis via inhibition of the pro inflammatory factor NFκB1 p50. In sum, these data provide evidence that statins inhibit the nuclear translocation of the inflammatory transcription factor NFkB1 p50 in vascular cells. These results suggest a mechanistic understanding for statin's pleiotropic benefits through their regulation of efferocytosis.

### HMG-CoA Reductase Inhibition Reduced the CD47 Expression in Human Atherosclerosis.

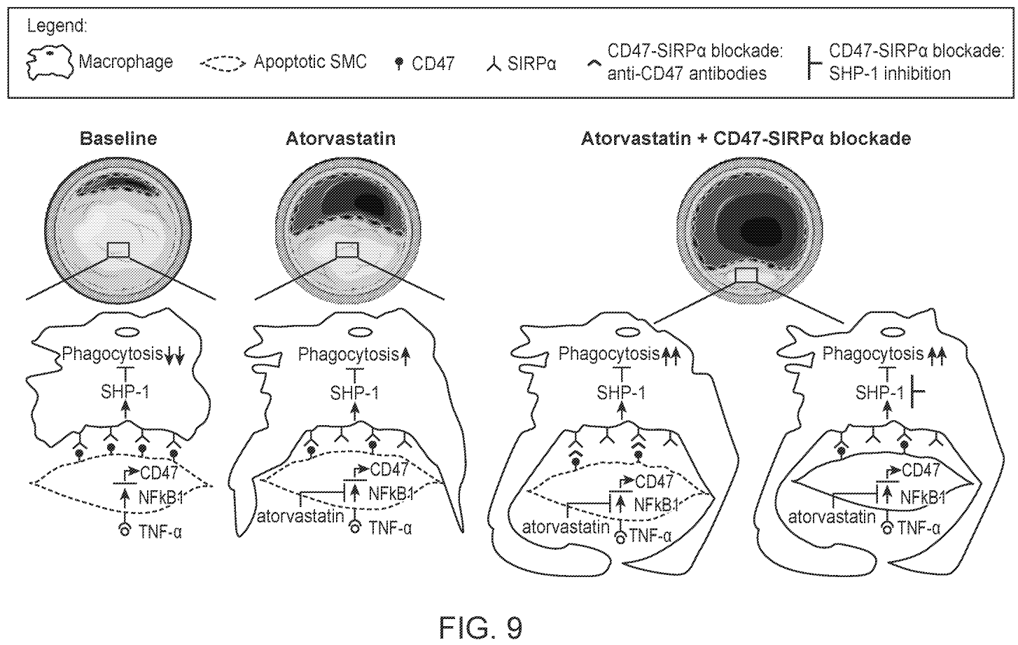

To determine if HMG-CoA reductase inhibitors result in lower CD47 expression during human atherogenesis, carotid endarterectomy samples were evaluated from the Munich Vascular Biobank. It was found that patients receiving statin treatment had lower CD47 expression than a propensity score matched cohort without such a medication (FIG. 8g). Taken together, these data suggested that HMG-CoA reductase inhibitor reduces the pathological upregulation of CD47 in human atherosclerosis and thus may have additive effects on the efferocytosis rate upon combined treatment (FIG. 9).

The foregoing studies provide new insights that can explain the pleiotropic effects of statins. It was shown that statins augment efferocytosis by inhibiting the nuclear translocation of NFκB1 p50 and suppressing expression of the key “don't eat me” molecule CD47. It was demonstrated that statins amplify the anti-atherosclerotic effects of two recently described pro-efferocytic therapies, and do so independent of any lipid-lowering effect. Analyses of clinical biobank specimens confirm a similar link between statins and CD47 expression in humans, highlighting the potential translational implication of these findings. These data provide a possible mechanism for how statins provide benefit beyond their well-described effect on cholesterol metabolism, and provide evidence that statins they may also reduce atherosclerosis by exerting a pro-phagocytic and anti-inflammatory effect directly in the vessel wall.

### Methods

### Bone Marrow-Derived Macrophages, Cell Sorting, RNA Sequencing Preparation, and Data Analysis

Bone marrow cells were isolated from C57BL/6J mice (The Jackson Laboratory) and differentiated ex vivo to macrophages in DMEM supplemented with 10% heat inactivated fetal bovine serum (FBS), 100 U/ml penicillin and 100 μg/ml streptomycin (HyClone GE HealthCare, SV30010), and 10 ng/ml murine M-CSF (Peprotech, Catalogue #315-02, Lot #0518245) for 7 to 10 days. After washing cells with pre-warmed PBS to remove non-attached cells, the attached primary mouse macrophages were incubated with 100 μM SWNT or SHP1i for 24 hours in serum-free medium at 37° C. After collecting and washing cells twice with 2% FBS-PBS, macrophages were sorted using a FACSAria cell sorter (BD Life Sciences, Stanford Shared FACS Facility). Channel compensations were performed using single-stained UltraComp eBeads (Thermo Scientific, Catalogue #01-2222-41) or control macrophages. In addition, macrophages were stained with SYTOX Blue (Invitrogen, Catalogue #S34837) to discriminate and exclude non-viable cells. Viable cells (SYTOX Blue negative) were sorted with a 100 μm nozzle into populations that were Cy5.5-positive and Cy5.5-negative and collected in 2% FBS-PBS. Then RNA was extracted using the miRNeasy Mini Kit (Qiagen, Catalogue #217004). The RNA samples were sent to Novogene Co. (Sacramento, CA, USA) for sample quality control, library preparation, and sequencing. All samples passed quality control. Subsequently, cDNA library construction and sequencing were performed for each sample on an Illumina Novaseq 6000 platform with paired-end 150 bp reads. The sequencing data were uploaded 1 to the Galaxy web platform, and we used the public server at usegalaxy.org to further analyse the data (version 2.0.1)15. Briefly, quality control of sequencing data was performed using FastQC. HISAT2 was used to map the reads to the reference genome (mm10). FeatureCounts was then used to count the number of reads mapped, and DESeq2 was used to generate the list of differentially regulated genes. P values were adjusted for multiple testing using Benjamin-Hochberg false discovery rate. Pathway and upstream regulator analyses were performed using Ingenuity Pathway Analysis (IPA, Qiagen).

### Animals and Diet

A total of 96 male apolipoprotein E-deficient (Apoe−/−) mice (B6.129P2-Apoe^{tm1Unc}/J, 002052) on a C56BL/6J background (The Jackson Laboratory) were used for this study: 9 animals in the PBS group, 10 animals in the atorvastatin group, 13 animals in the IgG group, 13 animals in the anti-CD47 group, 13 animals in the anti-CD47 plus atorvastatin group, 12 animals in the SWNT group, 11 animals in the SHP1i group, and 15 animals in the SHP1i plus atorvastatin group. Of note, lesion area of SHP1i animals compared to SWNT treated animals were published in our previous analysis (Flores et al., Nat. Nanotechnol. 15, 154-161, 2020). Animals were randomly assigned to the experimental groups and fed a high-fat diet (21% anhydrous milk fat, 19% casein, and 0.15% cholesterol, Dyets Inc.) for 2 weeks. For the ensuing 9 weeks on high-fat diet, mice then received the following therapies: (1) PBS by daily gavage versus atorvastatin (Lipitor, Pfizer, prescription formulation) at a dose of 10 mg/kg body weight per day by daily gavage (Jarr et al., Arterioscler Thromb Vasc Biol 40, 2821-2828, 2020); (2) 200 μg of the inhibitory anti-CD47 antibody (BioXCell, MIAP410, Catalogue #BE0283, Lot #705318N1) IP every other day versus 200 μg of the IgG1 isotype control (BioXCell, MOPC-21, Catalogue #BE0083, Lot #61991601 B) IP every other day (Kojima et al., Nature 536, 86-90, 2016); or (3) SWNT at a dose 1 of 200 μl of 400 nM IV once-weekly versus SHP1i at a dose of 200 μl of 400 nM IV once-weekly (Flores et al., Nat. Nanotechnol. 15, 154-161, 2020). Animal studies were approved by the Stanford University Administrative Panel on Laboratory Animal Care (protocol #27279) and conformed to the NIH guidelines for the care and use of laboratory animals.

### Tissue Preparation and Histological Analyses

Tissue preparation and histological analyses were performed as previously described (Kojima et al., Nature 536, 86-90, 2016; Jarr et al., Arterioscler Thromb Vasc Biol 40, 2821-2828, 2020). After blood sample collection, mice were perfused with PBS via cardiac puncture and then perfusion fixed with 4% phosphate-buffered paraformaldehyde. Blood samples were analyzed by the Stanford Animal Diagnostic Laboratory. The entire aortic arch was carefully collected, embedded in optimal cutting temperature compound (VWR, Catalogue #25608-930), and sectioned using a cryostat (Leica CM 1950). Plaque area (in % of total vessel area) was quantified by Oil-red O staining (Sigma-Aldrich, Catalogue #01516) and necrotic core (in % of lesion area) was quantified by Masson's trichrome staining (Richard-Allen Scientific, Catalogue #22-110-648). The necrotic core was defined as the neointimal area devoid of cellular tissue. For immunofluorescence staining of atherosclerotic lesions, cryosections were blocked using 5% goat serum (Sigma-Aldrich, Catalogue #G9023) in PBS. Next, sections were incubated overnight at 4° C. with the following primary antibodies: Mac3 (BD Life Sciences, Catalogue #550292, 1:100) and cleaved caspase-3 (Cell Signaling Technology, Catalogue #9661, 1:200). After extensive washing, sections were incubated with secondary antibodies from Thermo Scientific: Alexa Fluor 647 goat anti-rat (Catalogue #A-21247, Lot #2119156, 1:250) and Alexa Fluor 488 goat anti-rabbit (Catalogue #A11034, Lot #2110499, 1:250). Counterstaining to visualize nuclei was performed by incubating with DAPI (4′,6-diamidino-2-phenylindole). Histological sections were imaged using a Zeiss Axioplan (equipped with a Nikon camera) or Leica DMi8 microscope (equipped with a Leica DMC4500 colour camera). Fluorescence sections were imaged using a Leica DMi8 microscope (equipped with a Leica K5 camera). Sections were analysed using Image J/FIJI software (Version: 2.0.0/1.52p, NIH) in a blinded fashion.

### Bliss Independence Model

The Bliss independence model is a well-established method to determine additivity/synergy of compounds. The formula Ec=Ea+Eb−Ea·Eb, where Ec is the combined effect produced by the combination of compounds a and b, describes how a combination of compounds should act if no synergy exists16. We randomly shuffled the results of the single treatment groups using GraphPad random list generator. Then, we calculated Ec for each pair (referred to hereafter as Ecalculated) and compared these results to the observed results in the combined-treated cohort (referred to hereafter as Eobserved). A non-significant p value was considered to denote additivity.

### Cell Culture

Primary bone marrow-derived macrophages were grown in DMEM growth medium (Thermo Scientific, Catalogue #11995-065) supplemented with 10% heat-inactivated fetal bovine serum (Thermo Scientific, Catalogue #SH3007103H1), 100 U/ml penicillin, and 100 μg/ml streptomycin (HyClone GE HealthCare, Catalogue #SV30010). Mouse aortic vascular smooth muscle cells (Cell Biologics, Catalogue #C57-6080, Lot #M120919W12) were cultured and maintained according to the manufacturer's instructions. All cells were cultured in a humidified 5% CO2 incubator 1 at 37° C. The cell lines were authenticated by the supplier. None of the cell lines were tested for mycoplasma contamination. The following stimuli were applied to the cells in the experiments described below: atorvastatin (Sigma-Aldrich, Catalogue #PZ001, Source #0000040035, Batch #0000079529), Dimethyl sulfoxide (DMSO, sterile, Sigma-Aldrich, Catalogue #D2650), recombinant mouse tumor necrosis factor-α (TNF-α, aa 80-235, R&D systems, Catalogue #410-MT, Lot #CS1419081), DL-mevalonic acid 5-phosphate (Sigma-Aldrich, Catalogue #79849, Lot #BCBT1529), staurosporine (Sigma-Aldrich, Catalogue #S4400), anti-CD47 antibody (BioXCell, MIAP410, Catalogue #BE0283, Lot #792420D1), and IgG1 control (BioXCell, MOPC-21, Catalogue #BE0083, Lot #722919A2). When atorvastatin was used, equal concentrations of DMSO was added to all respective controls. Of note, the final concentration (v/v) of DMSO was equal or less than 0.1% to avoid toxic effects.

### In Vitro Phagocytosis Assay

Standard in vitro phagocytosis assays were performed using RAW 264.7 macrophages as phagocytes and target cells. Phagocytes were treated with 10 μM atorvastatin, 4 nM SHP1i, and equal concentrations of their respective controls (DMSO, SWNT) for 24 hours (in detail: “vehicle”=SWNT+DMSO; “Statin”=SWNT+atorvastatin; “SHP1i”=SHP1i+DMSO; “SHP1i+Statin”=SHP1i+atorvastatin). Apoptosis in target cells was induced by 1 μM staurosporine for 4 hours at 37° C. Additionally, target cells were labelled with 1.25 μM CellTracker Orange CMRA Dye (Thermo Scientific, Catalogue #C34551) according to the manufacturer's instructions. Phagocytes and target cells were then co-cultured for 2 hours at 37° C. Double positive cells (phagocytes=Cy5.5-positive, target cells=Orange-positive) were quantified using the LSRII (BD Life Sciences, Stanford Shared FACS Facility) and analysed by FlowJo10.7.1 (BD Life Sciences). Efferocytosis rate was defined as Q2 (double positive cells) divided by the sum of Q1 and Q2 (total number of apoptotic cells).

### Apoptosis Assay

The apoptosis assay was performed as previously described (Kojima et al., Nature 536, 86-90, 2016). To evaluate apoptosis, the luminometric Caspase-Glo 3/7 Assay System (Promega, Catalogue #G8091) was performed on cultured murine RAW 264.7 macrophages, according to the manufacturer's protocol. Cells were seeded in 96-well plates at the density of 10,000 cells per well, grown at 37° C. and serum-starved for 24 hours. Apoptosis was induced with 1 μM STS treatment for 4 hours in the presence or absence of 10 μM atorvastatin, 4 nM SHP1i, or equal concentrations of their respective controls (DMSO, SWNT). For quantification, an iD3 luminometer (Molecular Devices) was used.

### RNA Isolation and Quantitative Reverse-Transcription Polymerase Chain Reaction (PCR)

To measure Cd47 expression, mouse smooth muscle cells and murine bone marrow derived macrophages were exposed to DMSO, 10 μM atorvastatin, 50 ng/ml TNF-α+DMSO, or 50 ng/ml TNF-α+10 μM atorvastatin for 48 hours. To measure Apoe, Gpx3, Rbl1, Rhob, and Xiap expression, bone marrow-derived macrophages were exposed to DMSO or 10 μM atorvastatin for 48 hours. RNA was extracted from cell lysates using the miRNeasy Mini Kit (Qiagen, Catalogue #217004) according to the manufacturer's protocol or the TRIzol method (Invitrogen, Catalogue #15596026). Then, RNA was quantified with a NanoDrop One (Thermo Scientific). RNA was reverse transcribed using the High-Capacity RNA-to-cDNA Synthesis Kit (Applied 1 Biosystems, Catalogue #4387406). Quantitative PCR of the cDNA samples was performed on a ViiA7 Real-Time PCR system or a QuantStudio 5 (both Applied Biosystems). Gene expression levels were measured using TaqMan Universal Master Mix II (Applied Biosystems, Catalogue #4440047, Lot #00762728) and commercially available TaqMan primers (Applied Biosystems). Data were quantified with the 2^{-ΔΔCt} method and normalized to Gapdh as an internal control. The following TaqMan Primers were used: Cd47 (Mm00495011_m1), Apoe (Mm01307193_g1), Gpx3 (Mm00492427_m1), Rbl1 (Mm01250721_m1), Rhob (Mm00455902_m1), Xiap (Mm01311594_mH), and Gapdh (Mm99999915_g1).

### Flow Cytometry

To measure CD47 expression, mouse smooth muscle cells and bone marrow-derived macrophages were exposed to DMSO, 50 ng/ml TNF-α+DMSO, or 50 ng/ml TNF-α+10 μM atorvastatin for 48 hours. Cells were washed, harvested, and stained with an anti-CD47 antibody (BD Life Sciences, Catalogue #561890, FITC, MIAP301, 0.5 mg/ml) or an isotype control antibody (BD Life Sciences, Catalogue #553929, FITC, R35-95, 0.5 mg/ml) after Fc receptor blockade (BD Biosciences, Catalogue #553142, anti-mouse CD16/CD32). Expression was quantified using the LSRII (BD Life Sciences, Stanford Shared FACS Facility) and analysed by FlowJo10.7.1 (BD Life Sciences). The ratio of fluorescence intensity (RFI) was calculated by dividing the median fluorescence intensity of CD47 by the median fluorescence intensity of IgG isotype control.

### In Vitro Immunofluorescence

Mouse smooth muscle cells were seeded in Millicell EZ Slides 1 (Sigma-Aldrich, Catalogue #PEZGS0416 or Catalogue #PEZGS0816). For CD47 staining, cells were exposed to DMSO, 50 ng/ml TNF-α+DMSO, or 50 ng/ml TNF-α+10 μM atorvastatin for 48 hours. For NFκB1 p105/p50 staining, cells were first treated with DMSO, 10 μM atorvastatin, or 10 μM atorvastatin+100 μM mevalonate for 24 hours and then exposed to 50 ng/ml TNF-α for 45 minutes. Following stimulation/treatment, cells were rinsed with PBS and fixed with 4% phosphate-buffered paraformaldehyde. For CD47 staining (BioXCell, MIAP410, 25 μg/ml), vector mouse-on-mouse fluorescein Immunodetection Kit (Thermo Scientific, Catalogue #NC9801950) was used according to the manufacturer's instructions. For NFκB1 p105/p50 staining, cells were blocked with 5% goat serum (Sigma-Aldrich, Catalogue #G9023) for 30 minutes, then incubated with NFκB1 p105/p50 (Cell Signaling Technology, Catalogue #13586S, D4P4D, 1:200) overnight at 4° C. After extensive washing, cells were incubated with Alexa Fluor 594 goat anti-mouse (Thermo Scientific, Catalogue #A-11005, Lot #1696463, 1:300) or Alexa Fluor 647 goat anti-rabbit (Thermo Scientific, Catalogue #A-21244, Lot #56897A, 1:300), and DAPI (4′,6-diamidino-2-phenylindole). Images were captured using a Leica DMi8 microscope (equipped with a Leica DMC4500 colour camera and a Leica K5 camera for fluorescence imaging).

### Luciferase Reporter Assay

The luciferase reporter assay was performed as previously described (Kojima et al., Nature 536, 86-90, 2016). CD47 LightSwitch Promoter Reporter GoClones (RenSP, S710450) and Cypridina TK Control constructs (pTK-Cluc, SN0322S) were obtained from SwitchGear Genomics. 45 ng of the RenSP reporter and 5 ng of the pTK-Cluc reporter construct were transfected into mouse smooth muscle cells using Lipofectamine 3000 Transfection 1 Reagent (Thermo Scientific, Catalogue #L3000-008) and Opti-MEM I Reduced Serum Medium (Thermo Scientific, Catalogue #31985062). After 48 hours, media was changed to fresh medium and cells were then exposed to DMSO, 50 ng/ml TNF-α+DMSO, or 50 ng/ml TNF-α+10 μM atorvastatin. The cell lysate and supernatant were harvested 24 hours after stimulation/treatment und dual luciferase activity was measured with the LightSwitch Luciferase Assay Kit (Active Motif, Catalogue #32031, NC0999256) and Pierce Cypridina Luciferase Glow Assay Kit (Thermo Scientific, P116170) using an iD3 luminometer (Molecular Devices). Relative luciferase activity (RenSP/Cypridina ratio) was quantified as the percentage change relative to the basal value obtained from control-transfected cells.

### Protein Extraction and Western Blotting

To measure NFκB1 p50 nuclear translocation, mouse smooth muscle cells were first treated with DMSO, 10 μM atorvastatin, or 10 μM atorvastatin+100 μM mevalonate for 24 hours and then exposed to 50 ng/ml TNF-α for 45 minutes. Total protein was isolated from mouse smooth muscle cells using a subcellular protein fractionation kit (Thermo Scientific, Catalogue #78840) supplemented with Halt Protease and Phosphatase Inhibitor Cocktail (Thermo Scientific, Catalogue #78442). The protein concentration in each sample was measured using Pierce BCA Protein Assay Kit (Thermo Scientific, Catalogue #23225). Equal amounts of protein were loaded and separated on precast gels (Bio-Rad, Catalogue #456-1084) and thereafter transferred onto PVDF membranes (Life Technologies, Catalogue #LC2002). Following 1 hour incubation in 5% bovine serum albumin in 0.1% TBS-T, these membranes were probed with commercially available antibodies designed to recognize NFκB1 p105/p50 (Cell Signaling Technology, Catalogue #13586S, D4P4D, 1:1000) and HDAC1 (Cell Signaling Technology, Catalogue #5356S, 10E2, 1:1000) overnight at 4° C. After extensive washing with 0.1% TBS-T, membranes were incubated with secondary antibodies: Alexa Fluor 647 goat anti-mouse (Invitrogen, Catalogue #32728, Lot #TA252659, 1:10,000) and Alexa Fluor 488 goat anti-rabbit (Thermo Scientific, Catalogue #A11034, Lot #2110499, 1:10,000) for 1 hour. Membranes were then scanned with an iBright 1500 Imaging System (Thermo Scientific) for quantitative analysis using Image J/FIJI software (Version: 2.0.0/1.52p, NIH).

### Human Carotid Artery Tissue

The Munich Vascular Biobank contains human atherosclerotic plaques and plasma samples, along with clinical data obtained from patients receiving carotid endarterectomy. The authors state that their study complies with the Declaration of Helsinki, that the locally appointed ethics committee has approved the research protocol and that informed consent has been obtained from the subjects. In this study, a total of 14 human carotid endarterectomy samples were used as follows: age-, gender-, medication-, symptomatic-, and physical status-matched samples from 7 patients with statin medication were compared with 7 patients without such a medication (Source Data). Symptomatic stenosis was defined if the patient had suffered from carotid related symptoms, such as transient ischemic attack, amaurosis fugax, or stroke, within the last 6 months. Carotid tissue was cut in approximately 50 mg pieces on dry ice. Homogenization of the tissue was performed in 700 μl QIAzol lysis reagent and total RNA was isolated using the miRNeasy Mini Kit (Qiagen), according to the manufacturer's instruction. RNA concentration and purity were assessed using NanoDrop (Thermo Fisher Scientific). RNA integrity numbers for all samples were assessed using the RNA Screen Tape (Agilent) in the Agilent TapeStation 4200. Next, first strand cDNA synthesis was performed with the High-Capacity-RNA-to-cDNA Kit (Applied Biosystems), following the manufacturer's instruction. Gene expression levels were measured using commercially available TaqMan primers (Applied Biosystems): CD47 (Hs00179953_m1), and RPLP0 (Hs00420895_gH) on a QuantStudio 3 Cycler (Applied Biosystems) using 96 well plates.

### Statistical Analysis

Statistical analyses were performed using GraphPad Prism 9 (GraphPad Inc.). Continuous data are presented as mean (+/−standard error of the mean). Normality of data was determined by performing a D'Agostino and Pearson omnibus or Shapiro-Wilk normality test (α=0.05). Normally distributed data were analysed using an unpaired Student's t-test (two-tailed) and one-way analysis of variance with Tukey's multiple comparisons test. If samples had unequal variances (determined by F-test), an unpaired Welch's t-test (two-tailed) was used. For data that were not normally distributed, a Mann-Whitney U test (two-tailed) or a Kruskal-Wallis with Dunn's multiple comparisons test were used. A p value of 0.05 or less was considered to denote significance. All the data behind the statistical analysis and all p values are provided in Source data.

### Data Availability

Raw RNA sequencing data are available from the National Center for Biotechnology Information (NCBI) under accession number PRJNA7337400.

TABLE 3

| SEQ | | |
|---|---|---|
| ID | | |
| NO: | Description | Sequence |
| 1 | Human wild | MEPAGPAPGRLGPLLCLLLAASCAWSGVAGEEELQVIQPDKS |
| | type SIRP- | VLVAAGETATLRCTATSLIPVGPIQWFRGAGPGRELIYNQK |
| | alpha, | EGHFPRVTTVSDLTKRNNMDFSIRIGNITPADAGTYYCVKFR |
| | variant 1 | KGSPDDVEFKSGAGTELSVRAKPSAPVVSGPAARATPQHTVS |
| | | FTCESHGFSPRDITLKWFKNGNELSDFQTNVDPVGESVSYSIHST |
| | | AKVVLTREDVHSQVICEVAHVTLQGDPLRGTANLSETIRVPPTL |
| | | EVTQQPVRAENQVNVTCQVRKFYPQRLQLTWLENGNVSRTET |
| | | ASTVTENKDGTYNWMSWLLVNVSAHRDDVKLTCQVEHDGQP |
| | | AVSKSHDLKVSAHPKEQGSNTAAENTGSNERNIYIVVGVVCTL |
| | | LVALLMAALYLVRIRQKKAQGSTSSTRLHEPEKNAREITQDTN |
| | | DITYADLNLPKGKKPAPQAAEPNNHTEYASIQTSPQPASEDTLT |
| | | YADLDMVHLNRTPKQPAPKPEPSFSEYASVQVPRK |
| 2 | Human wild | MEPAGPAPGRLGPLLCLLLAASCAWSGVAGEEELQVIQPDKS |
| | type SIRP- | VLVAAGETATLRCTATSLIPVGPIQWFRGAGPGRELIYNQK |
| | alpha, | EGHFPRVTTVSDLTKRNNMDFSIRIGNITPADAGTYYCVKF |
| | variant 2 | RKGSPDDVEFKSGAGTELSVRAKPSAPVVSGPAARATPQHT |
| | | VSFTCESHGFSPRDITLKWFKNGNELSDFQTNVDPVGESVSYSI |
| | | HSTAKVVLTREDVHSQVICEVAHVTLQGDPLRGTANLSETIRV |
| | | PPTLEVTQQPVRAENQVNVTCQVRKFYPQRLQLTWLENGNVS |
| | | RTETASTVTENKDGTYNWMSWLLVNVSAHRDDVKLTCQVEH |
| | | DGQPAVSKSHDLKVSAHPKEQGSNTAAENTGSNERNIYIVVG |
| | | VVCTLLVALLMAALYLVRIRQKKAQGSTSSTRLHEPEKNAREI |
| | | TQDTNDITYADLNLPKGKKPAPQAAEPNNHTEYASIQTSPQPA |
| | | SEDTLTYADLDMVHLNRTPKQPAPKPEPSFSEYASVQVPRK |
| 3 | Human wild | MEPAGPAPGRLGPLLCLLLAASCAWSGVAGEEELQVIQPDKS |
| | type SIRP- | VLVAAGETATLRCTATSLIPVGPIQWFRGAGPGRELIYNQK |
| | alpha, | EGHFPRVTTVSDLTKRNNMDFSIRIGNITPADAGTYYCVKF |
| | variant 3 | RKGSPDDVEFKSGAGTELSVRAKPSAPVVSGPAARATPQHT |
| | | VSFTCESHGFSPRDITLKWFKNGNELSDFQTNVDPVGESVSYSI |
| | | HSTAKVVLTREDVHSQVICEVAHVTLQGDPLRGTANLSETIRV |
| | | PPTLEVTQQPVRAENQVNVTCQVRKFYPQRLQLTWLENGNVS |
| | | RTETASTVTENKDGTYNWMSWLLVNVSAHRDDVKLTCQVEH |
| | | DGQPAVSKSHDLKVSAHPKEQGSNTAAENTGSNERNIYIVVG |
| | | VVCTLLVALLMAALYLVRIRQKKAQGSTSSTRLHEPEKNAREI |
| | | TQDTNDITYADLNLPKGKKPAPQAAEPNNHTEYASIQTSPQPA |
| | | SEDTLTYADLDMVHLNRTPKQPAPKPEPSFSEYASVQVPRK |
| 4 | Human wild | MEPAGPAPGRLGPLLCLLLAASCAWSGVAGEEELQVIQPDKS |
| | type SIRP- | VLVAAGETATLRCTATSLIPVGPIQWFRGAGPGRELIYNQK |
| | alpha, | EGHFPRVTTVSDLTKRNNMDFSIRIGNITPADAGTYYCVKF |
| | variant 4 | RKGSPDDVEFKSGAGTELSVRAKPSAPVVSGPAARATPQHT |
| | | VSFTCESHGFSPRDITLKWFKNGNELSDFQTNVDPVGESVSYSI |
| | | HSTAKVVLTREDVHSQVICEVAHVTLQGDPLRGTANLSETIRV |
| | | PPTLEVTQQPVRAENQVNVTCQVRKFYPQRLQLTWLENGNVS |
| | | RTETASTVTENKDGTYNWMSWLLVNVSAHRDDVKLTCQVEH |
| | | DGQPAVSKSHDLKVSAHPKEQGSNTAAENTGSNERNIYIVVG |
| | | VVCTLLVALLMAALYLVRIRQKKAQGSTSSTRLHEPEKNAREI |
| | | TQVQSLDTNDITYADLNLPKGKKPAPQAAEPNNHTEYASIQTS |
| | | PQPASEDTLTYADLDMVHLNRTPKQPAPKPEPSFSEYASVQVP |
| | | RK |
| 5 | Human wild | EEELQVIQPDKSVSVAAGESAILHCTVTSLIPVGPIQWFRGAGP |
| | type d1 | ARELIYNQKEGHFPRVTTVSESTKRENMDFSISISNITPADAGTY |
| | domain of | YCVKFRKGSPDTEFKSGAGTELSVRAKPS |
| | SIRP-alpha | |
| 6 | mutant d1 | EEELQIIQPDKSVLVAAGETATLRCTITSLFPVGPIQWFRGAGPG |
| | domain of | RVLIYNQRQGPFPRVTTVSDTTKRNNMDFSIRIGNITPADAGTY |
| | SIRP-alpha | YCIKFRKGSPDDVEFKSGAGTELSVRAKPS |
| 7 | mutant d1 | XXELQVIQPDKSVLVAAGETATLRCTATSLIPVGPIQWFRGAG |
| | domain of | PGRELIYNQKEGHFPRVTTVSDLTKRNNMDFSIRIGNITPADAG |
| | SIRP-alpha | TYYCVKFRKGSPDDVEFKSGAGTELSVR |
| 8 | mutant d1 | XXELQVIQPDKSVSVAAGESAILHCTVTSLIPVGPIQWFRGAGP |
| | domain of | ARELIYNQKEGHFPRVTTVSESTKRENMDFSISISNITPADAGTY |
| | SIRP-alpha | YCVKFRKGSPDTEFKSGAGTELSVR |
| 9 | mutant d1 | EEXLQVIQPDKXVXVAAGEXAXLXCTXTSLIPVGPIQWFRGAG |
| | domain of | PXRELIYNQKEGHFPRVTTVSXXDLTKRXNMDFXIXIXNITPAD |
| | SIRP-alpha | AGTYYCVKFRKGSPDDXEFKSGAGTELSVR |
| 10 | Human wild | EEELQVIQPDKSVSVAAGESAILHCTVTSLIPVGPIQWFRGAGP |
| | type d1 | ARELIYNQKEGHFPRVTTVSESTKRENMDFSISISNITPADAGTY |
| | domain of | YCVKFRKGSPDTEFKSGAGTELSVRAKPSDKTHTCPPCPAPEL |
| | SIRP-alpha | LGGPSVFLFPPKPKDTLMISRTPEVTCVVVDVSHEDPEVKFNW |
| | fused to an | YVDGVEVHNAKTKPREEQYNSTYRVVSVLTVLHQDWLNGKE |
| | IgG1 | YKCKVSNKALPAPIEKTISKAKGQPREPQVYTLPPSRDELTKNQ |
| | sequence | VSLTCLVKGFYPSDIAVEWESNGQPENNYKTTPPVLDSDGSFF |
| | | LYSKLTVDKSRWQQGNVFSCSVMHEALHNHYTQKSLSLSPGK |
| 11 | Human wild | EEELQVIQPDKSVSVAAGESAILHCTVTSLIPVGPIQWFRGAGP |
| | type d1 | ARELIYNQKEGHFPRVTTVSESTKRENMDFSISISNITPADAGTY |
| | domain of | YCVKFRKGSPDTEFKSGAGTELSVRAKPSESKYGPPCPPCPAPE |
| | SIRP-alpha | FLGGPSVFLFPPKPKDTLMISRTPEVTCVVVDVSQEDPEVQFN |
| | fused to an | WYVDGVEVHNAKTKPREEQFNSTYRVVSVLTVLHQDWLNGK |
| | IgG4 | EYKCKVSNKGLPSSIEKTISKAKGQPREPQVYTLPPSQEEMTKN |
| | sequence | QVSLTCLVKGFYPSDIAVEWESNGQPENNYKTTPPVLDSDGSF |
| | | FLYSRLTVDKSRWQEGNVFSCSVMHEALHNHYTQKSLSLSLG |
| | | K |
| 12 | dl domain | EEELQIIQPDKSVLVAAGETATLRCTITSLFPVGPIQWFRGAGPG |
| | of CV1- | RVLIYNQRQGPFPRVTTVSDTTKRNNMDFSIRIGNITPADAGTY |
| | hlgG4 or | YCIKFRKGSPDDVEFKSGAGTELSVRAKPS |
| | CV1 | |
| | monomer | |
| 13 | dl domain | EEELQIIQPDKSVLVAAGETATLRCTITSLFPVGPIQWFRGAGPG |
| | of CV1 | RVLIYNQRQGPFPRVTTVSDTTKRNNMDFSIRIGNITPADAGTY |
| | fused to the | YCIKFRKGSPDDVEFKSGAGTELSVRAKPSAAAPPCPPCPAPEF |
| | human IgG4 | LGGPSVFLFPPKPKDTLMISRTPEVTCVVVDVSQEDPEVQFNW |
| | Fc domain | YVDGVEVHNAKTKPREEQFNSTYRVVSVLTVLHQDWLNGKE |
| | (i.e. CV1- | YKCKVSNKGLPSSIEKTISKAKGQPREPQVYTLPPSQEEMTKN |
| | hlgG4) | QVSLTCLVKGFYPSDIAVEWESNGQPENNYKTTPPVLDSDGSF |
| | | FLYSRLTVDKSRWQEGNVFSCSVMHEALHNHYTQKSLSLSPG |
| | | K |
| 14 | dl domain | EEELQIIQPDKSVLVAAGETATLRCTITSLFPVGPIQWFRGAGPG |
| | of CV1 | RVLIYNQRQGPFPRVTTVSDTTKRNNMDESIRIGNITPADAGTY |
| | fused to the | YCIKFRKGSPDDVEFKSGAGTELSVRAKPSAAAVECPPCPAPP |
| | human IgG2 | VAGPSVFLFPPKPKDTLMISRTPEVTCVVVDVSHEDPEVQFNW |
| | Fc domain | YVDGMEVHNAKTKPREEQFNSTFRVVSVLTVVHQDWLNGKE |
| | (i.e. CV1- | YKCKVSNKGLPAPIEKTISKTKGQPREPQVYTLPPSREEMTKN |
| | hlgG2) | QVSLTCLVKGFYPSDIAVEWESNGQPENNYKTTPPMLDSDGSF |
| | | FLYSKLTVDKSRWQQGNVFSCSVMHEALHNHYTQKSLSLSPG |
| | | K |
| 15 | dl domain | EEEVQIIQPDKSVSVAAGESAILHCTITSLFPVGPIQWFRGAGPA |
| | of FD6- | RVLIYNQRQGPFPRVTTISETTRRENMDFSISISNITPADAGTYY |
| | hlgG4 or a | CIKFRKGSPDTEFKSGAGTELSVRAKPS |
| | FD6 | |
| | monomer | |
| 16 | dl domain | EEEVQIIQPDKSVSVAAGESAILHCTITSLFPVGPIQWFRGAGPA |
| | of FD6 | RVLIYNQRQGPFPRVTTISETTRRENMDESISISNITPADAGTYY |
| | fused to the | CIKFRKGSPDTEFKSGAGTELSVRAKPSAAAPPCPPCPAPEFLG |
| | human IgG4 | GPSVFLFPPKPKDTLMISRTPEVTCVVVDVSQEDPEVQFNWYV |
| | Fc domain | DGVEVHNAKTKPREEQFNSTYRVVSVLTVLHQDWLNGKEYK |
| | (i.e. FD6- | CKVSNKGLPSSIEKTISKAKGQPREPQVYTLPPSQEEMTKNQVS |
| | hlgG4) | LTCLVKGFYPSDIAVEWESNGQPENNYKTTPPVLDSDGSFFLY |
| | | SRLTVDKSRWQEGNVFSCSVMHEALHNHYTQKSLSLSPGK |
| 17 | dl domain | EEEVQIIQPDKSVSVAAGESAILHCTITSLFPVGPIQWFRGAGPA |
| | of FD6 | RVLIYNQRQGPFPRVTTISETTRRENMDFSISISNITPADAGTYY |
| | fused to the | CIKFRKGSPDTEFKSGAGTELSVRAKPSAAAVECPPCPAPPVAG |
| | human IgG2 | PSVFLFPPKPKDTLMISRTPEVTCVVVDVSHEDPEVQFNWYVD |
| | Fc domain | GMEVHNAKTKPREEQFNSTFRVVSVLTVVHQDWLNGKEYKC |
| | (i.e. FD6- | KVSNKGLPAPIEKTISKTKGQPREPQVYTLPPSREEMTKNQVSL |
| | hIgG2) | TCLVKGFYPSDIAVEWESNGQPENNYKTTPPMLDSDGSFFLYS |
| | | KLTVDKSRWQQGNVFSCSVMHEALHNHYTQKSLSLSPGK |

1. A method of reducing vascular inflammation in a human subject, the method comprising:
administering to the subject an effective dose of an anti-CD47 agent; and
monitoring the subject for indicia of vascular inflammation.

2. The method of claim 1, wherein the method is performed in the absence of genotyping the subject for the presence of at least one 9p21 risk allele.

3. The method of claim 1, wherein the anti-CD47 agent specifically binds to CD47.

4. The method of claim 1, wherein the anti-CD47 agent is an antibody that does not activate CD47 upon binding, optionally comprising an IgG4 constant region.

5-6. (canceled)

7. The method of claim 3, wherein the anti-CD47 agent is a soluble SIRPα polypeptide, optionally, wherein the SIRPa polypeptide is selected from a CV1-hIgG4, CV1 monomer, FD6-hIgG4; a FD6 monomer, or the polypeptides in Table 3.

8. The method of claim 7, wherein the soluble SIRPα polypeptide comprises an immunoglobulin constant region, optionally multimerized through the immunoglobulin constant region.

9. (canceled)

10. The method of claim 1, wherein the anti-CD47 agent is administered to the subject at a dose of 20-45 mg/kg weekly.

11. The method of claim 1, wherein the anti-CD47 agent is administered to the subject weekly for at least nine weeks.

12. The method of claim 1, further comprising administering a priming dose of the anti-CD47 agent to the subject prior to administering the therapeutically effective dose of the anti-CD47 agent to the subject, optionally at a dose of 1 mg/kg.

13. (canceled)

14. The method of claim 1, wherein vascular inflammation is reduced by at least 10%, or at least 20%.

15. (canceled)

16. The method of claim 1, wherein the indicia of vascular inflammation is selected from the group consisting of a change in vascular ¹⁸F-FDG uptake, high sensitivity C-reactive protein (hsCRP), C-reactive protein (CRP), IL-6, IL-8, fibrinogen, Human serum amyloid A (SAA), Haptoglobin (Hp), secretory phospholipase A2 (sPLA2), Lipoprotein(a), apolipoprotein B (APOB) to apolipoprotein A1 (APOA1) ratio, and white blood cell count (WBC).

17. The method of claim 16, wherein the indicia of vascular inflammation is a change is vascular ¹⁸F-FDG uptake, optionally as measured by maximum standardized update values (SUV) and/or maximum target-to-background ratio (TBR) or by combined Positron Emission Tomography (PET) and computed tomography (CT);
wherein vascular ¹⁸F-FDG uptake is reduced by at least 10% or by at least 20%.

18-21. (canceled)

22. A method of reducing vascular inflammation in a human subject, the method comprising:
administering to the subject an effective dose of an anti-CD47 agent in combination with an effective dose of a statin, wherein the combination provides for a reduction in vascular inflammation relative to the effect of either agent as a monotherapy.

23. The method of claim 22, wherein the reduction in vascular inflammation is additive or synergistic relative to the effect of either agent as a monotherapy.

24. (canceled)

25. The method of claim 22, wherein the statin is selected from atorvastatin, fluvastatin, lovastatin, mevastatin, pitavastatin, pravastatin, rosuvastatin, and simvastatin.

26. The method of claim 22, wherein the method is performed in the absence of genotyping the subject for the presence of at least one 9p21 risk allele.

27. The method of claim 22, wherein the anti-CD47 agent specifically binds to CD47.

28. The method of claim 27, wherein the anti-CD47 agent is an antibody that does not activate CD47 upon binding, optionally comprising an IgG4 constant region.

29-30. (canceled)

31. The method of claim 27, wherein the anti-CD47 agent is a soluble SIRPα polypeptide, optionally, wherein the SIRPa polypeptide is selected from the polypeptides in Table 3.

32. The method of claim 31, wherein the soluble SIRPα polypeptide comprises an immunoglobulin constant region, optionally multimerized through the immunoglobulin constant region.

33-35. (canceled)

36. The method of claim 31, wherein the SIRPa polypeptide is selected from a CV1-hIgG4, CV1 monomer, FD6-hIgG4 or a FD6 monomer, or from the polypeptides in Table 3.

37. The method of claim 22, wherein the reduction in vascular inflammation results in a plaque area as a measure of total vessel area is reduced by at least 5% compared to the absence of intervention, or wherein the reduction in vascular inflammation results in a necrotic core as a measure of the percentage of intima area is reduced by at least 5% compared to the absence of intervention.

38. (canceled)

39. The method of claim 22, wherein the reduction in vascular inflammation results in an increased rate of efferocytosis, optionally wherein the rate of efferocytosis is increased by at least 10% compared to the absence of intervention.

40. (canceled)


SEQUENCE LISTING (17 sequences)

SEQ ID NO: 1 - protein, 504 aa, Homo Sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - protein, 504 aa, Homo Sapiens; identical to SEQ ID NO: 1

SEQ ID NO: 3 - protein, 504 aa, Homo Sapiens; identical to SEQ ID NO: 1

SEQ ID NO: 4 - protein, 508 aa, Homo Sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 5 - protein, 118 aa, Homo Sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 6 - protein, 119 aa, Homo Sapiens
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 7 - protein, 115 aa, Homo Sapiens
  misc_feature (1)..(2): Xaa can be any naturally occurring amino acid
XXELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVR

SEQ ID NO: 8 - protein, 114 aa, Homo Sapiens
  misc_feature (1)..(2): Xaa can be any naturally occurring amino acid
XXELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVR

SEQ ID NO: 9 - protein, 117 aa, Homo Sapiens
  misc_feature (3)..(3): Xaa can be any naturally occurring amino acid
  misc_feature (12)..(12): Xaa can be any naturally occurring amino acid
  misc_feature (14)..(14): Xaa can be any naturally occurring amino acid
  misc_feature (20)..(20): Xaa can be any naturally occurring amino acid
  misc_feature (22)..(22): Xaa can be any naturally occurring amino acid
  misc_feature (24)..(24): Xaa can be any naturally occurring amino acid
  misc_feature (27)..(27): Xaa can be any naturally occurring amino acid
  misc_feature (45)..(45): Xaa can be any naturally occurring amino acid
  misc_feature (65)..(66): Xaa can be any naturally occurring amino acid
  misc_feature (72)..(72): Xaa can be any naturally occurring amino acid
  misc_feature (77)..(77): Xaa can be any naturally occurring amino acid
  misc_feature (79)..(79): Xaa can be any naturally occurring amino acid
  misc_feature (81)..(81): Xaa can be any naturally occurring amino acid
  misc_feature (104)..(104): Xaa can be any naturally occurring amino acid
EEXLQVIQPDKXVXVAAGEXAXLXCTXTSLIPVGPIQWFRGAGPXRELIYNQKEGHFPRVTTVSXXDLTKRXNMDFXIXIXNITPADAGTYYCVKFRKGSPDDXEFKSGAGTELSVR

SEQ ID NO: 10 - protein, 345 aa, Homo Sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 347 aa, Homo Sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 119 aa, Homo Sapiens; identical to SEQ ID NO: 6

SEQ ID NO: 13 - protein, 346 aa, Homo Sapiens
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSAAAPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 345 aa, Homo Sapiens
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSAAAVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 118 aa, Homo Sapiens
EEEVQIIQPDKSVSVAAGESAILHCTITSLFPVGPIQWFRGAGPARVLIYNQRQGPFPRVTTISETTRRENMDFSISISNITPADAGTYYCIKFRKGSPDTEFKSGAGTELSVRAKPS

SEQ ID NO: 16 - protein, 345 aa, Homo Sapiens
EEEVQIIQPDKSVSVAAGESAILHCTITSLFPVGPIQWFRGAGPARVLIYNQRQGPFPRVTTISETTRRENMDFSISISNITPADAGTYYCIKFRKGSPDTEFKSGAGTELSVRAKPSAAAPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 17 - protein, 344 aa, Homo Sapiens
EEEVQIIQPDKSVSVAAGESAILHCTITSLFPVGPIQWFRGAGPARVLIYNQRQGPFPRVTTISETTRRENMDFSISISNITPADAGTYYCIKFRKGSPDTEFKSGAGTELSVRAKPSAAAVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK